(19) United States
(12) Patent Application Publication
(10) Pub. No.: US 2011/0203007 A1
(43) Pub. Date: Aug. 18, 2011

(54) ASSAYS OF NEURODEGENERATIVE DISORDERS, INCLUDING FRONTOTEMPORAL DEMENTIA AND AMYOTROPHIC LATERAL SCLEROSIS

(75) Inventors: Ronald Klein, Shreveport, LA (US); Phillip Henning, Shreveport, LA (US); David Wang, Shreveport, LA (US); Robert Dayton, Shreveport, LA (US); Jason Tatom, Shreveport, LA (US); Elysse Orchard, Shreveport, LA (US)

(73) Assignee: BOARD OF SUPERVISORS OF LOUISIANA STATE UNIVERSITY AND AGRICULTURAL AND MECHANICAL COLLEGE

(21) Appl. No.: 12/706,642

(22) Filed: Feb. 16, 2010

(51) Int. Cl. A01K 67/027; A61K 49/00; A61K 38/16; A61K 31/7088; A61P 25/28

(52) U.S. Cl. 800/3; 800/14; 800/21; 514/44 R; 514/17.7

(57) ABSTRACT

The invention relates to novel assays for the in vivo analysis of neurodegenerative diseases and the use of such assays to discover therapies capable of modulating such diseases.

### FIELD OF THE INVENTION

The invention relates to novel assays for the in vivo analysis of neurodegenerative diseases and the use of such assays to discover therapies capable of modulating such diseases.

### BACKGROUND OF THE INVENTION

Despite great effort, there is a lack of treatment options for most neurodegenerative diseases. The current lack of an effective therapy for neurodegenerative diseases, such as frontotemporal lobar degeneration (FTLD) and amyotrophic lateral sclerosis (ALS), could be linked to the lack of in vivo assays to study both underlying mechanisms of these diseases and the subsequent drug development based on these assays. Improvements in animal assays will unveil translatable drug candidates that may miss or may not translate when derived from otherwise existing in vitro or in vivo assays. In other words, assays with greater predictive validity than neurotoxins, germ-line transgenics, or in vitro approaches are worth pursuing. It is contemplated herein that, with improvements as outlined below, the vector based assays of human neurodegenerative diseases have utility as a stage for screening drug targets; and that both the assays themselves and a new drug can be validated. The vector based assays disclosed herein could provide understanding of disease processes, and help lead to a useful drug.

Numerous methods of gene transfer are known in the art, and are not reviewed in any great detail here. Suffice it to say that in general, methods of gene transfer in vitro are well known and have been practiced for several decades. Methods of in vivo gene transfer are much more recent, but have been successfully applied in such contexts as gene therapy efforts to overcome genetic disorders, and in disease modeling efforts, such as the production of germ-line transgenic animal models, such as gene knockout mice or transgenic mice and other animals expressing heterologous genes. These assays have led to the generation of animal based assays of human disease, providing a better understanding of the disease and a mechanism to identifying therapies useful in treating the disease. For a global review of ALS and other Neurodegenerative Disorders see Neurodegenerative Dementias:Clinical Features and Pathological Mechanisms, (edited by Christopher Clark and John Trojanowski), McGraw-Hill (2000); and Mitochondrial Inhibitors and Neurodegenerative Disorders, (edited by Paul Sandberg et al.) Humana Press (2000).

In general, the known methods of in vivo gene transfer involve the knockout of single genes present in the genome of an animal model, or the inclusion in the germ-line of a specific transgene in the genome of an animal model. The limitations to such methods include the possibility of inducing terminal illnesses in the animal assays, such that either non-viable fetuses are produced, or limited life-span animals are produced. In addition, the effects of multiple gene knockouts or transgenes are extremely difficult to simulate in such systems, due to the complex temporal, gene regulatory and interaction effects in such systems. Furthermore, the germ-line transgenic models currently available tend to provide data on a very slow time scale, and such efforts as drug modeling and disease analysis are delayed by the time-scale of transgenic animal maturation. Recently, a method of somatic gene transfer was developed that allowed for the modeling of human diseases in rodents (3-6), including many human neurodegenerative diseases. These applications and patent specifically described the uses of tau, α-synuclein, presenelin and APP to create such animal models. The present invention describes a novel assay for frontotemporal disorders, including ALS, through gene transfer of the TDP-43 gene.

TDP-43 or TAR DNA Binding Protein is a normal cellular protein of 43 kDa that was originally discovered as the cellular protein involved in binding the transactivating region of HIV DNA that contains RNA binding motifs (7, 8). TDP-43 has also been demonstrated to play an important roll in the development of cystic fibrosis through its involvement in the splicing of the CFTR gene (9, 10). More recently, TDP-43 was found in ubiquitinated cytoplasmic and neuritic inclusions in the neurons from affected regions of patients with frontotemporal lobar degeneration and ALS (1, 11). It has also been found to be present in non-ubiquitinated glial occlusions in one case of familial frontotemporal dementia (12). The absence of genetic linkage in a Manchester cohort of frontotemporal dementia patients, suggests that the protein accumulation may be a consequence of the disease (13); however, others have not ruled out a causative role in the disease. TDP-43 has also been found to accumulate in the brains of Guam Parkinson's dementia patients (14), hippocampal sclerosis (15), neurodegenerative diseases including Lewy bodies (16), Alzheimer's disease (17), Pick's disease (18), and has been proposed for use as a diagnostic for neurodegenerative disorders, including Alzheimer's disease (19). While all of this has been intriguing, the true role of TDP-43 in the brain and its relevance to the various diseases remain uncertain (20, 21), although recently a number of mutations have been discovered linking TDP-43 to neurodegenerative diseases (22). The creation of transgenic animals represents an important means for studying the role of TDP-43 in these diseases. The development of animal assays of these diseases, using the wild-type TDP-43 or any of the TDP-43 mutants in a vector capable of delivering the gene to the brains of animals, so that the role of TDP-43 in the pathology of the disease can be assessed would be very helpful. Such assays can also serve as a means of screening for or testing therapies being developed for treating these diseases.

In their landmark paper, Neumann et al., (2006) screened antibodies for binding ubiquitinated, taunegative lesions of cases of FTLD, including a familial mutation in progranulin (Baker et al., 2006), and were able to identify the substrate protein, TDP-43. The protein is normally in the nucleus, but disease neuropathology involves its aberrant cytoplasmic mislocalization (Neumann et al., 2006). Neumann et al. then solved the mysterious pathology of non-SOD mutation ALS, the same cytoplasmic TDP-43 neuropathology as in non-tau FTLD. It turns out, neuropathology in FTLD-TDP and ALS have much in common, including TDP-43 cytoplasmic inclusions in the hippocampus and the substantia nigra (Neumann et al., 2009; Amador-Ortiz et al., 2007; Geser et al., 2008). While FTLD-TDP and ALS overlap in the mapping distribution of TDP-43 pathology, patients manifest either motoric (ALS) or central (FTLD-TDP) dysfunction first (Geser et al., 2009). There is a variant of FTLD-TDP called FTLD-motor neuron disease, with both symptoms, and ALS patients may progressively develop FTLD symptoms (Geser et al., 2009), so there is a shared pattern of pathology in ALS and FTLD-TDP. Not too surprisingly, TDP-43 pathology may overlap with the other classic neuropathologies of beta-amyloid, alpha-synuclein, and tau: estimates of 23-56% of AD (Amador-Ortiz et al., 2007; Uryu et al., 2008; Arai et al., 2009), 31% of dementia with Lewy bodies (Nakashima-Yasuda et al., 2007), and two families of FTLD-TDP with progranulin mutations with mixed TDP-43/tau pathology (Leverenz et al., 2007). There are a number of papers describing cases of TDP-43 pathology in the substantia nigra or striatum along with parkinsonism (Geser et al., 2008; reviewed in Tatom et al., 2009), and TDP-43 pathology is now well known in hippocampal sclerosis (Josephs & Dickson, 2007; Amador-Ortiz, 2007; 2007a). Therefore, a blocker of TDP-43 toxicity could therefore have broad relevance from FTLD-TDP to ALS, and maybe even cases of AD and parkinsonism with TDP-43 pathology as well.

As TDP-43 pathology is throughout the CNS in FTLD and ALS (Geser et al. 2009; 2009), it would be beneficial to improve the mimicry by systemic AAV9 gene delivery for widespread TDP-43 expression in the brain and spinal cord.

### SUMMARY OF THE INVENTION

At the present time there is no known treatment for diseases involving TDP-43 proteinopathies. Accordingly, the present invention relates to the fields of novel assays for the study of neurodegenerative diseases and the use of such to discover therapies capable of modulating the disease. More specifically, this invention describes the use of viral delivery of TDP-43 to create assays of diseases involving TDP-43. Such assays may include models of frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS).

TDP-43 was identified by Neumann et al. (1) as the protein composing the filamentous neuropathological lesions in ALS and FTLD-U. This has established TDP-43 as a hallmark neuropathological substrate along with amyloid, tau, synuclein, and polyglutamine. It is therefore contemplated that the assays disclosed herein could have broad significance for ALS considering all cases of ALS have TDP-43 lesions in the spinal cord and the brain, save the ALS cases harboring superoxide dismutase mutations, which is 2% of ALS (2). This invention also describes the use of such assays to identify therapies useful in treating these diseases.

The invention features animal assays of neurodegenerative disorders involving TDP-43 alone or in combination with SOD, tau and/or α-synuclein. These assays can provide a way to better test the efficacy of new chemical entities. Such assays can also be used to study and gain a better understanding of the mechanisms leading to these neurodegenerative disorders. Additionally, as new gene products are discovered that interact with TDP-43 using in vitro screening assays or based on in vivo observations, the function of these new TDP-43 interacting gene products can be analyzed by generating animal assays involving TDP-43 in combination with one or more of the interacting gene products. Such gene products could be proteins, peptides, mRNA or miRNAs.

In one aspect the invention features a method for creating animal assays of neurodegenerative diseases, including but not limited to frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS), by delivery of the TDP-43 gene into the brain of rodents. This method includes placing the TDP-43 gene into an expression vector such that the protein product is expressed in the animal such as a rodent. The vector can be either a plasmid or viral vector, preferably the adeno-associated virus. The expression of the TDP-43 gene can be controlled by a gene promoter. Once the vector has been constructed, the gene is introduced into the brain and the TDP-43 protein is expressed. Following a period of time allowing sufficient accumulation of the protein, the assays can be used to study TDP-43 neuropathies or to screen therapeutic regimens, such as small molecules or other therapeutic agents, for their efficacy in treating such diseases.

Assays for studying neurodegenerative diseases of varying severity are also provided. For example, the severity of the disease in the assays disclosed herein can be controlled by adjusting the concentration of gene introduced to the animal and/or introducing the gene into an animal of a certain age. For example, when the gene is introduced into a rat at day 1, the rat displays a more severe phenotype than a rat wherein the gene is introduced at day 2. It is contemplated that this is due to the maturation of the blood brain barrier. The AAV can pass the blood brain barrier for up until a few days after birth, and the AAV9 is an especially good type for this (Foust et al.). It is known that mannitol is a blood brain barrier disrupting agent and that, when administered with the AAV vector, it allows entry of the AAV vector with reporter genes. In older and adult animals, it is contemplated that mannitol may be utilized to temporarily disrupt the blood brain barrier (McCarty et al.). Therefore hearty, longer living animal based assays can be provided by introducing the gene at day 2-3 rather than at day 1. One can thus study purely adult onset neurodegenerative diseases by employing a mannitol/vector combination, which can be introduced either sequentially or together with the gene. In some embodiments, the mannitol is introduced from about 5 minutes to about 10 minutes prior to the introduction of the gene.

In the forgoing aspect of the invention, the vector used to insert the TDP-43 gene is the adeno-associated virus (AAV) serotype 9 vector and the TDP-43 gene is the human TDP-43 wild-type gene. Human TDP-43 genes containing mutations, either mutations found in individuals or mutations of interest to a researcher can be used, as can other vectors.

Further, in the forgoing method, the promoter used to drive expression of TDP-43 is the cytomegalovirus/chicken β-actin promoter. Other promoters can also be used that lead to constitutive, inducible or cell-specific expression of the TDP-43 gene product.

In the foregoing methods, the vector can be introduced by injection into the substantia nigra, injection into the hypoglossal nucleus, or by intravenous delivery. In certain embodiments, the vector is not introduced injection into the hypoglossal nucleus. Other methods of introducing a vector include use of a gene gun, liposomes, and the ex vivo gene therapy approach. When the foregoing method of sterotaxic delivery to the substantia nigra of the AAV-9 vector containing the human TDP-43 gene under the control of the cytomegalovirus/chicken β-actin promoter is used, expression of the protein is noted by seven days post-injection and persists at least 30 days post-injection.

While the early, disclosed findings are in young rats, the AAV system could be applied to various strains of mice and various transgenic mouse lines, as well as other appropriate animal assays for neurodegenerative research such as other types of rodents and non-human primates. While young rats were targeted, application to aged subjects is included, as well as AAV gene transfer to very young neonatal animals, to produce widespread gene transfer throughout the brain. This approach could mimic germ-line transgenic mouse lines in terms of the spread of the gene transfer. The rat substantia nigra was initially targeted because of its disease relevance, although the system could be applied to any part of the central or peripheral nervous system to mimic TDP-43 diseases, such as ALS where targeting motor neurons in the spinal cord and brain is relevant. A retrograde targeted delivery system could also be applied, where the AAV particle could infect a neuronal axon in the periphery, or a long distance away within the brain, and be retrogradely trafficked to the cell body for TDP-43 expression targeted to the innervating area, e.g., in the nigrostriatal system, injection into the striatum, the axonal area, for targeted expression in neuronal cells of the substantia nigra, the innervating area. This strategy would allow for study of TDP-43 without a needle injection site in the region of interest, as well as for a distal delivery system to the CNS from the periphery. In one embodiment, the gene delivery method disclosed herein involves slow infusion into the brain parenchyma (tissue) through a stainless steel injection cannula.

The initial study of wild-type TDP-43 gene transfer was targeted to the substantia nigra which is relevant to diseases with TDP-43 pathology and parkinsonism as mentioned above, although the facility to study neuronal loss and behavioral effect in rats by lesioning the nigrostriatal pathway was also a practical issue. Most of the human TDP-43 was expressed in neuronal nuclei, while only a small percentage of the transduced cells (1-2%) displayed the human-like pathology of cytoplasmic TDP-43. The cells with cytoplasmic expression did resemble human samples (Neumann et al., 2006), and were ubiquinated, so it is possible to achieve good mimicry in some cells when overexpressing wild type TDP-43.

Disclosed herein are methods for improving the mimicry to the TDP-43 disease most widely affecting the population, ALS, by: 1) directing expression to the cytoplasm; 2) targeting upper and lower motor neurons in the spinal cord and brain; and 3) expressing a C-terminal TDP-43 fragment that is found in disease samples. The AAV vector system has potential for the growing field of TDP-43, as the innate toxicity of this nuclear protein has likely slowed the development of transgenic mice (or rats) up till now (Xu et al., 2008; Cleveland, 2009; Igaz et al., 2009; Stallings et al., 2009; Zhou et al., 2009). However, an important new transgenic mouse has just recently surfaced using a familial mutant form of TDP-43 (Wegorzewska et al., in press). There are other excellent systems used to study TDP-43 expression as well such as yeast (Johnson et al., 2008), Drosophila (Feiguin et al., 2009; Hazelett et al., 2009), C. elegans (Kraemer et al., 2009), zebrafish (Van Damme, 2009; Laird et al., 2009), and chick embryos (Sreedharan et al., 2008). Using the methods disclosed herein, a AAV/rat model is provided which can be useful for researching FTLD-TDP and ALS.

In addition, compounds capable of altering the neuronal effects of TDP-43 can be tested in the foregoing assays. A difference in neuronal number, as determined, for example, by histological evaluation of brain sections, or a change in neuronal function, as determined, for example, by PET analysis of brain metabolism, or a change in behavior, for example rotational behavior, would indicate drug efficacy or the potential importance of an interacting gene product. A therapeutically effective amount is, for example, a dosage sufficient to prevent or reverses the TDP-43 expression or accumulation without significant side effects or toxicities. The compound may be in a pharmaceutically acceptable carrier.

In another aspect, the invention features the additional step of combining the TDP-43 gene with SOD, tau and/or α-synuclein. The combination of TDP-43 with one or more of these other genes can result in assays of other neuropathies. Such new assays can also be used to assess the efficacy of small molecules for treating that particular proteinopathy.

In yet another aspect, the invention features methods for developing a better understanding of a particular proteinopathy by combining TDP-43 with genes coding for products that interact with TDP-43.

In the foregoing aspect of the invention, the identified genes and/or products can be used directly as a gene therapy, in the case of genes, or the protein, peptide or miRNA products of the gene can themselves be used, to prevent the deleterious effects of TDP-43 on neuronal function and fecundity. Such genes or gene products would be identified using this invention by demonstrating their ability to prevent or reverse the histochemical, functional or behavioral deficits caused by TDP-43 expression. The gene or gene product could then be used at a therapeutically effective concentration to treat the disease.

In the foregoing aspect of the invention, the identification and validation in the assays described in this invention using TDP-43 interacting genes provide a strong basis for the development of new diagnostics. Such genes, when shown to exasperate the proteinopathy in the assays described in this invention, could be analyzed by a quantifiable method, such as ELISA analysis, in patients suspected of possessing neuropathies.

This invention allows the rat/vector system to bridge towards drug development, to deliver assays ready for gene and drug target validation. There are several aspects disclosed herein which are highly relevant to different TDP-43 diseases: 1) investigation of TDP-43 and memory function; 2) animal assays of dysarthria, i.e., bulbar onset ALS; 3) intravenous AAV9 gene delivery for spinal onset ALS assay; 4) gene therapy with wild type progranulin, a gene linked to FTLD-TDP when mutated. One aspect of the present invention uses intravenous AAV9, with the goal of disease modeling in animals (e.g. rats).

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a Western Blot of TDP-43 overexpression in the SN at 2 weeks post-injection. The TDP-43 AAV9 consistently upregulated the 43 kDa band relative to GFP AAV9 control vector, uninjected (U) or vehicle (V) injected tissues. There was a 5.6 fold increase in the expression of TDP-43 in the TDP-43 treated animals compared with GFP treated animals. Three subjects for either vector group are shown, which all received an equivalent dose of 1×10¹⁰ vector genomes.

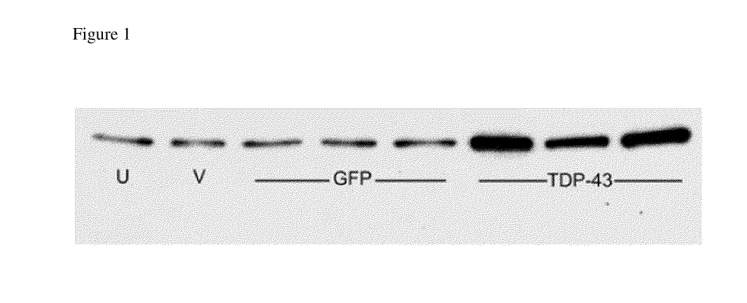

FIG. 2 demonstrates deposition of rat ubiquitin after human TDP-43 gene transfer, in both cytoplasmic and nuclear aggregates. A-D) Examples of ubiquitin positive inclusions in the TDP-43 vector group, which were not found in control vector transduced or non-transduced tissues (not shown). Three week time point. A-D, bar=10 μm.

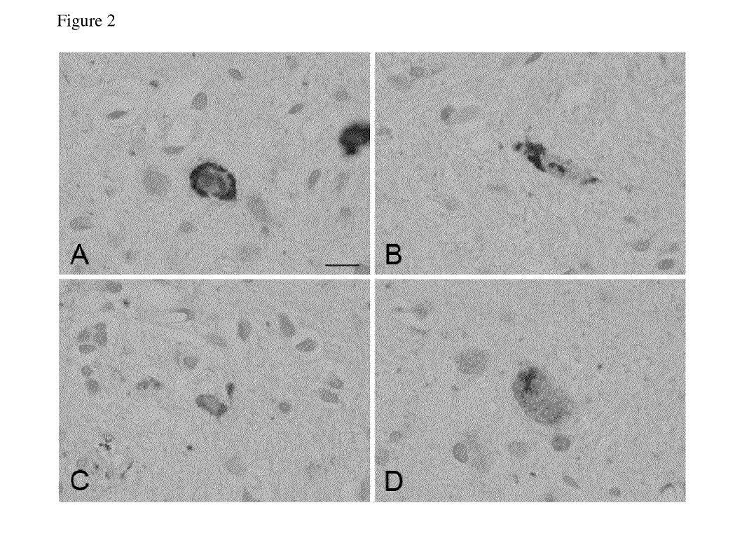

FIG. 3 demonstrates microglial staining with antibody Cd11b in animals treated with a fixed dose of 1×10¹⁰ vector genomes (vg), of either AAV9 GFP (A-E) or AAV9 TDP-43 (F-J). TDP-43 obviously induced microgliosis relative to GFP, especially away from the needle track in the substantia nigra, with apparent increases in staining at intervals 3-7 days, 7-14 days, and 14-28 days, the longest time point studied. Bar=180 μm.

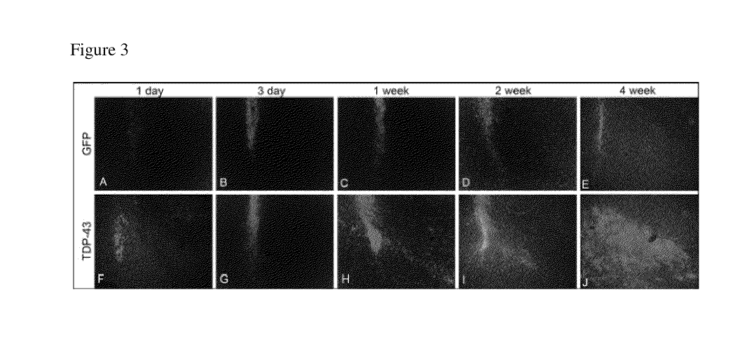

FIG. 4 shows dose-dependent AAV TDP-43 induced microgliosis (Cd11b antibody). Rats were treated with doses of either 1×10¹⁰ or 3×10¹⁰ vector genomes (vg) and the interval was 4 weeks. At both doses, microglial staining was elevated away from the needle track in the TDP-43 group (C, D), relative to GFP (A, B), and there was elevated staining in the high dose TDP-43 group (D) relative to the low dose group (C). Bar=125 μm.

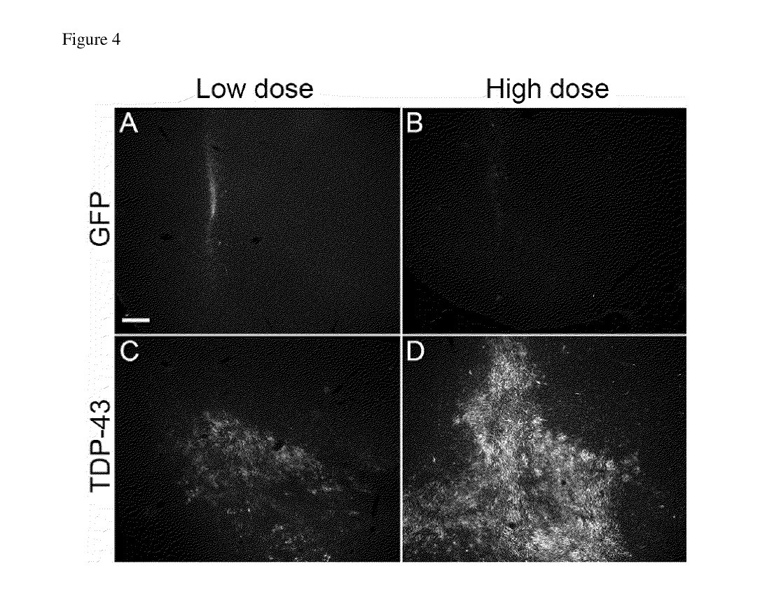

FIG. 5 demonstrates that TDP-43 gene transfer reduces tyrosine hydroxylase immunoreactivity in the substantia nigra (SN). A) TH labeling in the SN pars compacta in uninjected tissue. B) Four weeks after a dose of 3×10¹⁰ vector genomes (vg) of the GFP AAV9 vector, there was no change in TH staining. Section in A is from the contralateral side of B. C, D) Contralateral uninjected, and TDP-43 AAV9 transduced tissue, injected at a dose of 1×vg. E, F) Contralateral uninjected, and TDP-43 AAV9 transduced tissue, injected at a dose of 3×10vg. There was partial and more complete lesioning of the SN with TDP-43 gene transfer, depending on the dose. A-F, bar=100 μm.

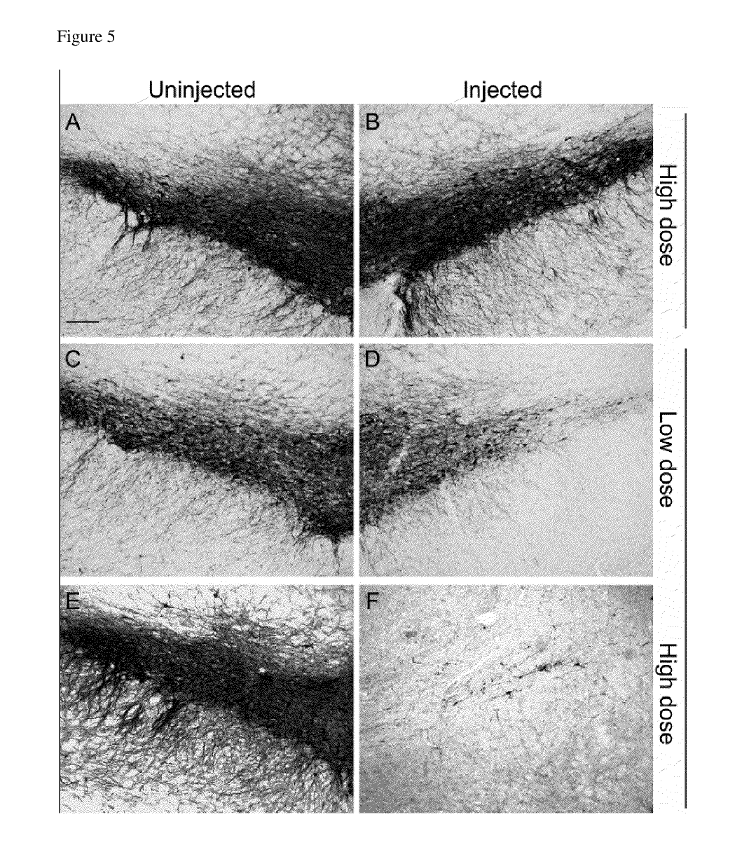

FIG. 6 demonstrates that TDP-43 gene transfer reduces tyrosine hydroxylase immunoreactivity in the striatum (4 weeks). For each subject, the right side is the uninjected side and the left side received either GFP or TDP-43 vector at 2 doses (low dose 1×10¹⁰ vector genomes; high dose 3×10¹⁰ vector genomes). While GFP vector injections did not produce side-to-side differences in the density of TH axons in the striatum (A, B), the TDP-43 injections appeared to reduce fiber density relative to the contralateral side, and in a dose-dependent manner (C, D). A-D, bar=610 μm.

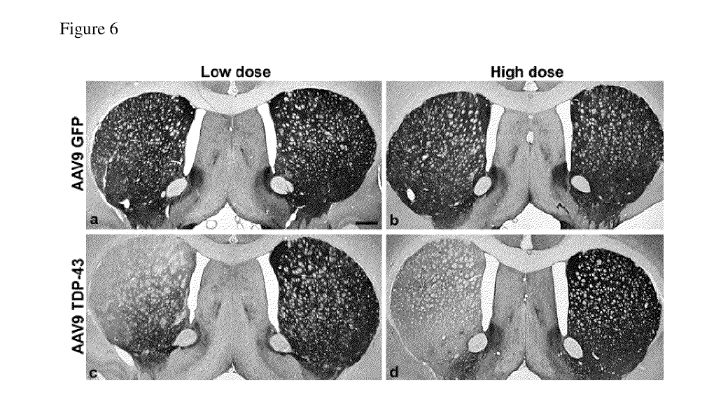

FIG. 7 shows progressive development of amphetamine-stimulated rotational behavior after high dose TDP-43 gene transfer. A) GFP AAV9 low dose (1×10¹⁰ vector genomes, vg) group, tested at 2 and 4 weeks. B) GFP AAV9 high dose (3×10¹⁰ vg). C) TDP-43 AAV9 low dose as in A. D) TDP-43 AAV9 high dose as in B. There was a significant ipsilateral turning bias only in the high dose TDP-43 group, and at 4, but not 2 weeks. *, P<0.05, t-test.

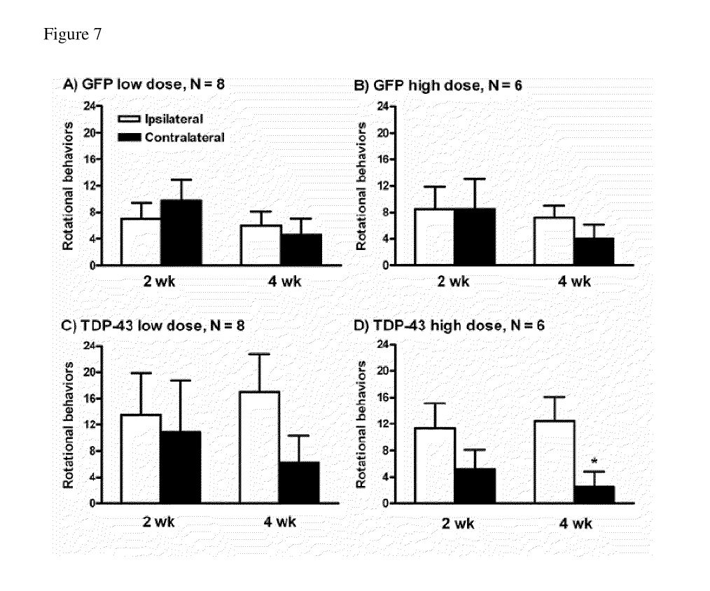

FIG. 8 shows cytoplasmic expression of TDP-43 and ubiquitination by the host rat. A, B, C) Examples of cells in the rat substantia nigra with cytoplasmic TDP-43 pathology (arrows) when human TDP-43 was expressed with AAV9. D) The control group only had nuclear TDP-43 staining and no cytoplasmic staining. The antibody used in A-D recognizes both rat and human TDP-43. The cytoplasmic TDP-43 expression was diffuse and granular, indicating an early stage of pathology at this 4 week interval. E, F) H & E staining showed abiotrophic, pyknotic cells in the TDP-43 group (F), and only healthy cells in the control group (E, arrows). Consistent with the H & E results, apoptotic nuclei were confirmed by TUNEL staining in the TDP-43 samples (not shown). G, H) In the areas of human TDP-43 expression, there were cells with cytoplasmic ubiquitin deposition in the TDP-43 group (H), but not in controls (G). There is good mimicry of human TDP-43 pathology in rats, at least in some of the cells overexpres sing TDP-43. I-K) Confocal double labeling of human TDP-43 (I, red) and beta (III) tubulin, a neuronal marker (J, green). Not only was the vector-derived TDP-43 found in the nucleus, as expected, but also dotted along the plasmalemma of TDP-43 transduced neurons (Tatom et al., 2009).

FIG. 9 shows rats injected with AAV9 intravenously one day after birth and viewed 28 days later. A) A control GFP rat displays the normal hindlimb extension escape response when raised by its tail. B) Among several obvious phenotypes in a TDP-43 rat, the hindlimbs cross to the midline (center) when raised and there is drastic loss of muscle tone in the hindlimbs (right).

FIG. 10 shows AAV9 transgene expression 32 days after injections to one day old rat pups. A) Efficient GFP expression (immunofluorescence) in the thalamus, in cells with both neuronal and glial morphology, in a GFP rat. B) Cortical neurons expressing GFP. C) Human specific TDP-43 immunofluorescence in a high dose TDP-43 rat.

FIG. 11 shows GFP immunofluorescence in spinal cord of a GFP rat at 32 days. The image in A is overexposed to show GFP in the ventral spinal cord. The cuneate and gracile fasciculi, the corticospinal tract, and lamina of the dorsal horn are enriched in GFP fibers, and there are a few large motor neurons in the ventral horn expressing GFP. It is likely that both ascending and descending sensory and motor neurons were transduced. (See The Spinal Cord: A Christopher and Dana Reeve Foundation Text and Atlas (Academic Press)).

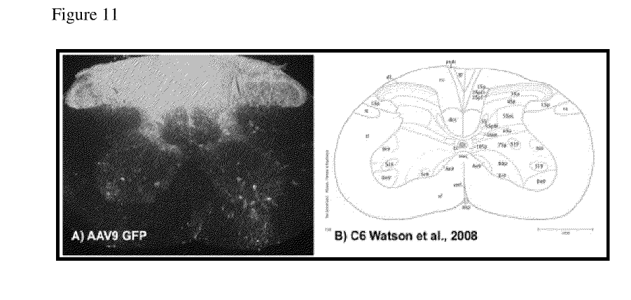

FIG. 12 shows a time-course of TDP-43 induced microgliosis. Staining for microglia with antibody Cd11b at intervals after control AAV9 GFP gene transfer to the substantia nigra, mainly labeled the needle track, whereas after AAV9 TDP-43, Cd11b immunoreactivity increased away from the needle track between 1-4 weeks, minoring the rise in transgene expression (Tatom et al., 2009).

FIG. 13 shows ALS relevant muscle wasting in TDP-43 treated animals. A) Atrophy of anterior tibialis (A.T.), medial gastrocnemius (M.G.), and soleus (Sol.) in a TDP-43 rat relative to a control GFP rat. B) Hematoxylin & eosin stain of paraffin section of medial gatrocnemius from a GFP rat. C) The myofibers are severely atrophied in a TDP-43 rat (same magnification as B). The smaller and angulated myofibers indicate end stage dennervation, reminiscent of amyotrophic lateral sclerosis and other dennervation myopathies.

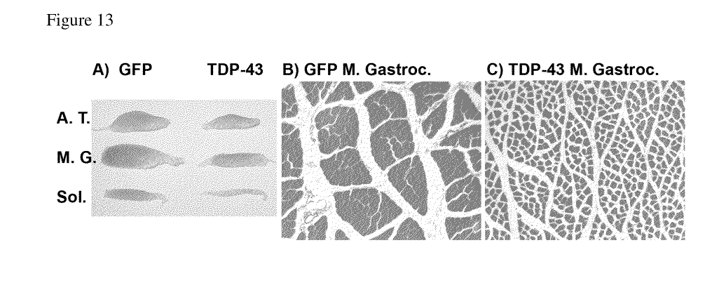

FIG. 14 shows the impairment of locomotor function in TDP-43 treated animals. A, B) A control subject ambulates on all four paws and rears (stands up) normally. C, D) The TDP-43 subject drags its hindlimbs and its abdomen touches the floor, and it cannot fully rear.

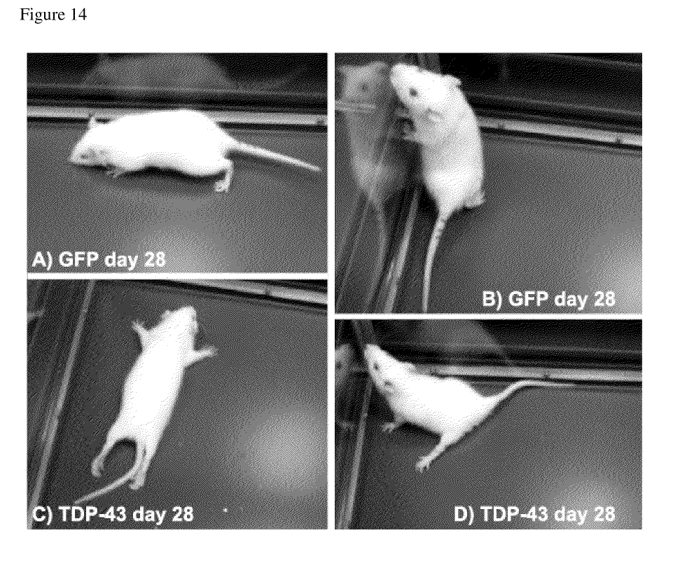

FIG. 15 shows the loss of motor control and performance in TDP-43 animals. Rats were tested for ability to stay on an accelerating wheel (4-40 RPM over 2 min), before the time to fall off. GFP rats performed similarly to untreated age-matched rats (one month old), while the TDP-43 group had paresis (partial paralysis) of the hindlimbs, preventing good performance, and mimicking the loss of motor control in ALS. Statistical difference by t test (p<0.003; n=5-6 per group).

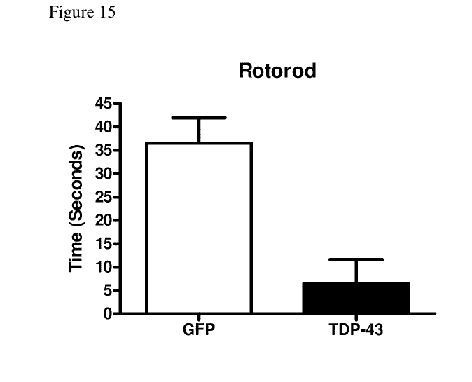

### DETAILED DESCRIPTION OF THE INVENTION

Before the present compositions and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “a promoter” includes a plurality of promoters and equivalents thereof known to those skilled in the art.

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5% or 1%.

By “compound” or “small molecule” is meant a chemical, be it naturally-occurring or artificially-derived, that is screened by employing one of the assay methods described herein. Candidate compounds may include, for example, peptides, polypeptides, synthetic organic molecules, naturally occurring organic molecules, nucleic acid molecules, sugars, polysaccharides, and derivatives thereof.

By “gene” is meant any chain of two or more deoxyribonucleic acids that encode a gene product when expressed in a cell.

By “gene product” is meant the RNA transcribed from a gene, whether the RNA is a coding RNA such as mRNA or a non-coding RNA such as miRNA and the protein or peptide product resulting from the translation of the mRNA. The gene products can be synthesized naturally within a cell or made synthetically.

By “miRNA” is meant a microRNA or a single strand RNA molecule of about 21-23 nucleotides in length that regulates gene expression by binding to complementary sequences in mRNAs.

By “mRNA” is meant any chain of nine or more ribonucleic acids that when translated result in the production of a protein or peptide.

By “neurodegenerative disease” is meant a disease that is caused by or results in neuronal deficits in the brain. For example, diseases such as frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS) are all examples of neurodegenerative disease.

By “promoter” is meant a regulatory region of DNA located upstream of a gene which regulates the transcription of said gene. The promoter can be a conventional promoter or a selective promoter, such as a motor neuron specific promoter.

By “protein” or “peptide” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “proteinopathies” is meant a disease involving non-normal protein.

By “therapeutically effective amount” is meant an amount of a compound sufficient to produce a preventative, healing, curative, stabilizing, or ameliorative effect in the treatment of a condition, e.g., a proteinopathy.

By “transgene” or “transgenic” is meant an exogenous gene introduced into another organism. The AAV system is an example of somatic cell gene transfer, i.e. to non germ-line cells. The somatic transgenic system is typically more localized than germ-line transgenic mice. Other somatic cell systems such as lentivirus vectors, adenovirus vectors, herpes virus vectors, including non-viral systems such as naked DNA and DNA complexed with any type of known carrier molecules including nanoparticles and modified nanoparticles, or encapsulated DNA, could be applied to express TDP-43 by known methods in the field. The non-viral methods could be of use in mimicking the glial pathology in TDP-43 diseases because viral vectors such as AAV are more neurotropic. However, a glial specific promoter in the context of both AAV and non-viral approaches could target glial TDP-43 pathology (e.g., glial fibrillary acidic protein promoter for astrocytes, myelin basic protein for oligodendrocytes and Schwann cells).

By “treating” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

By “vector” is meant any agent that acts as a carrier or transporter, as a virus or plasmid that conveys a genetically engineered DNA segment into a host cell.

An “expressible gene construct” refers to genetic material comprising at least a promoter, a vector or plasmid and the gene such as TDP-43 which is desired to be expressed in the non-human animal wherein the construct is capable of expressing the genetic products encoded by the gene in vivo. The expressible gene construct can also include other genetic material such as the gene for progranulin, tau, parkin, SOD, etc. Alternatively, two or more expressible gene constructs can be administered to the non-human animal.

A “disease marker” refers to a condition or a set of conditions associated with one or more neurodegenerative disease. For example, dementia can be associated with non-Alzheimer's dementia, dementia lacking distinctive histopathology, and the like.

The term “human TDP-43” refers to both wild-type human TDP-43 as well as mutant human TDP-43, including either naturally occurring mutants or researcher-derived mutants.

### Models

TDP-43 has been shown to be associated with SOD in frontotemporal dementia and ALS (23). In certain clinical cases, TDP-43 has been demonstrated to be associated with α-synuclein (17) and tau (24). The nuclear protein TDP-43 was found to be the neuropathological substrate in non-tau forms of FTLD and ALS and other neurodegenerative diseases (Neumann et al., 2006; Amador-Ortiz et al., 2007; Uryu et al., 2008; Arai et al., 2009; Neumann, 2009). The present invention provides methods and assays for TDP-43 to more comprehensively model FTLD. Disclosed herein are TDP-43 gene transfer methods for inducing TDP-43 neurodegeneration. When TDP-43 is overexpressed it is quite toxic to neurons, which may explain the slow development of TDP-43 transgenic mice up to now. TDP-43 gene transfer with AAV9 has provided unequivocal examples of cytoplasmic and ubiquinated lesions, apoptosis, micro- and astrogliosis, vector dose-dependent loss of dopaminergic neurons in the substantia nigra and their axons in the striatum, progressive motoric behavior deficit, and expression of TDP-43 in neuronal plasmalemma.

Targeting the substantia nigra in rats, it is shown that the tau pathology and parkinsonism associated with subtypes of FTLD-tau (progressive supranuclear palsy, corticobasal degeneration, frontotemporal dementia with parkinsonism associated with chromosome 17) (Klein et al., 2004; Uhbi et al., 2009; Jaworski et al., in press; Osinde et al., 2008). Also disclosed herein are methods for targeting regions of the CNS: hippocampus, upper motor neurons in the brain, lower motor neurons in the brainstem and spinal cord. In addition to tau-induced parkinsonism in FTLD, other neurodegenerative diseases such as ALS, and non-tau FTLD can be studied using the assays disclosed herein.

Thus, another aspect of this invention is to use TDP-43 in combination with SOD, tau and/or α-synuclein to create animal based assays of the neurodegenerative diseases for the study of disease processes or identification of therapies.

TDP-43 diseases include FTLD, ALS, AD and hippocampal sclerosis. Tau or TDP-43 neuropathology are involved in FTLD. While the role of tau in AD and FTLD-tau is well known, TDP-43 is more novel, with the field expanding quickly after the discovery that it is the pathological hallmark protein of about fifty percent of FTLD cases, and of the vast majority of ALS (Neumann et al., 2006; Neumann, 2009; Geser et al., 2009). Symptomologies are commonly shared among subclasses of FTLD, ranging from changes in behavior and personality, dementia, language deficits and aphasia, motor defects and parkinsonism, and typically leading to death within a short period, on the order of a decade (Cairns et al., 2007; Kumar-Singh and Van Broekhoven, 2007; Neumann, 2009). However, the ubiquitinated neuropathological lesions found postmortem may be: 1) ubiquinated neurofibrillary tangles rich in tau; or 2) ubiquitinated cytoplasmic or nuclear inclusions rich in TDP-43; or 3) ubiquitinated inclusions of a protein called FUS (Neumann et al., 2009; Lagier-Tourenne and Cleveland, 2009). The form of FTLD with the ubiquitinated lesions is rich in TDP-43 (also known as FTLD-ubiquitin) (Cairns et al., 2007; Kumar-Singh and Van Broekhoven, 2007; Neumann, 2009), but a logical nosology of FTLD-tau, -TDP, and -FUS is being adopted (Neumann, 2009; Neumann et al., 2009).

Disclosed herein are assays for FTLD, wherein said assays are provided via gene transfer of the TDP-43 pathological substrate protein. The gene transfer approach disclosed herein can also be applied for the TDP-43 pathology in motor neurons in ALS (Kwong et al., 2007; Geser et al., 2009; 2009a), a devastating degenerative disease devoid of good medication. TDP-43 is significant for the vast majority of ALS, except rare familial forms involving mutations in superoxide dismutase (SOD), which is estimated to be 2% of the patient population (Mackenzie et al., 2007). This has led to a shift in focus away from SOD and towards TDP-43 in the ALS field. It is thus contemplated that the methods and assays disclosed herein provide research tools relevant to 98% of ALS, and 50% of FTLD. There are pathological and symptomological spectra for FTLD ranging from tau to TDP-43, and for TDP-43 pathology ranging from FTLD-TDP to ALS and AD.

With TDP-43 expression targeted to other brain regions associated with ALS and FTLD, such as the motor cortex or hypoglossal nucleus or hippocampus, very relevant behavioral syndromes in the rat are contemplated. For example, targeting TDP-43 induced apoptosis to upper motor neurons could mimic the paralysis of ALS and loss of hypoglossal neurons will introduce loss of control of the tongue like the dysarthria that occurs in bulbar onset (brainstem rather than spinal cord) ALS.

This invention describes animal based assays of human neuropathies generated by transferring a construct capable of expressing the human TDP-43 gene into the brains of rodents. The construct can be introduced to the rodent by injection into the substantia nigra, injection into the hypoglossal nucleus, by infusion or by intravenous administration. In certain embodiments, the construct is not introduced injection into the hypoglossal nucleus. Said invention could be practiced using either the wild-type human TDP-43 gene or mutant TDP-43 genes, either naturally occurring mutants or reserarcher-derived mutants. Assays for identifying compounds for use in treating or preventing a neuropathic disease associated with TDP-43, including diseases such as frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS), are described herein.

These assays involve identifying histochemical, functional or behavioral changes in the TDP-43 animal assay following treatment with compounds. The identified compounds can potentially be used in the treatment of neuropathic diseases, such as frontotemporal dementias or ALS, or in other diseases with TDP-43 over expression. These TDP-43 rodent assays can also be used to study the disease progression. In addition, other genes, such as SOD, tau and ci-synuclein, which have been shown to associate with TDP-43, can be combined with TDP-43 in an animal assay to identify small molecules capable of treating neuropathies or to gain a better understanding of the disease of interest. Similarly, the genes for gene products demonstrated to interact with TDP-43 can be combined with TDP-43 in the animals to generate assays for screening small molecules or studying disease mechanisms and progression. The information resulting from such combination may also lead to the development of novel diagnostics.

### Vector Construction

This invention requires an expression vector to deliver TDP-43 and/or other genes into the brains of rodents. The expression can be driven through the use of a constitutively active promoter such that the gene product is expressed in every cell taking up the vector; an inducible promoter such that the gene product is expressed only when induced by an external stimulus; or a cell-specific promoter such that the gene product is expressed in only a specific cell type of interest, preferably dopaminergic cells. Such promoters are known to those skilled in the art, but by way of example, a constitutively active promoter could be the promoters from elongation factor 1 alpha or a viral promoter such as CMV, SV40 or RSV, or chicken β-actin; examples of inducible promoters include the metalathion, heat shock protein, tetracycline or minocycline promoters; and examples of cell-specific promoters include those from human synapsin-1 promoter and compact glial fibrillary acidic protein promoter.

As an example, the human wild-type TDP-43 cDNA was purchased from a commercial source (Invitrogen) and moved into the appropriated plasmid for AAV packaging and expression. The plasmid contained the AAV2 terminal repeats, the CMV/chicken beta-actin promoter, and the bovine growth hormone polyadenylation sequence. We cross-packaged the AAV2 terminal repeat plasmid into AAV9 virions by published methods (25) using the 2 other needed helper/packaging plasmids for AAV9. The AAV9 was made by cell transfection, purified on iodixanol gradient, and washed and concentrated by described methods (25). Many other methodologies could be applied for TDP-43 gene transfer vectors, including other AAV serotype variants, other known purification systems, and other disease-related familial mutation variants of the TDP-43 (e.g. (26)).

The vector may be chosen based on the desired injection site of the therapy. For example, for intravenous administration, the vector must be capable of accessing the brain of the animal and therefore must be capable of crossing the blood-brain barrier. AAV9 may be used for intravenous administration.

Similar vectors can be used to deliver not only the wild-type TDP-43 gene but also mutant TDP-43 genes. Likewise, the vectors, either the same vector used to deliver TDP-43 or a different vector, can be used to deliver the combinations of genes. Additionally, two or more genes may be delivered on a single vector containing two or more promoters. When any combination of two or more genes are delivered using a single vector or two or more vectors, the promoters driving expression of the genes can be identical or different. For example, a constitutive promoter may be used to drive expression of TDP-43 and an inducible promoter to drive expression of the interacting gene product.

### Vector Delivery

In addition to the infusion parameters described herein, known techniques such as convection-enhanced delivery or use of various injection cannulae including pulled glass pipettes, could be applied for TDP-43 gene transfer. For ALS assays, delivery to the spinal cord and motor related areas of the CNS will be targeted. For global gene transfer, neonatal rodent pups could be infused intracerebroventricularly or intrathecally.

As an example, Male Sprague-Dawley rats (3 or 20 months old, from Harlan, Indianapolis, Ind.) were anesthetized with a cocktail of 3 ml xylazine, 3 ml ketamine, and 1 ml acepromazine administered intramuscularly at a dose of 1 ml/kg. Viral stocks were injected through a 27 gauge cannula connected via 26 gauge internal diameter polyethylene tubing to a 10 μl Hamilton syringe mounted to a microinjection pump at a rate of 0.2 μl/min. The stereotaxic injection coordinates for substantia nigra in 3 month old rats were found to be, from Bregma: 5.3 mm posterior, 2.1 mm lateral, 7.6 mm ventral with 1.5 μl injected at each depth for 3 month old rats. Through a trial and error process the coordinates in 20 month old rats were found to be, from Bregma: 6.2 mm posterior, 2.1 mm lateral, 7.8 mm ventral with 1.5 μl injected at each depth. The needle remained in place at each injection site for 1 additional min before the cannula was removed slowly. The skin was sutured, and the animal was observed until it began to recover from the surgery, before being returned to its home cage.

### Generation of TDP-43 Rodent Assay

Disclosed herein is an assay using male Sprague-Dawley rats, which were injected in the substantia nigra (SN) with adeno-associated virus (AAV) serotype 9 expressing human TDP-43. TDP-43 lesions occur in the SN in ALS with dementia (27), and in FTLD (24). This region includes a population of dopamine neurons, which can be quantified in rats to test for TDP-43 induced dopaminergic neurodegeneration.

The AAV9 TDP-43 gene transfer led to clear expression of the transgene (over 5-fold endogenous levels) for periods between 1 week and 1 month, the period tested. Statistics back effects of TDP-43 on loss of dopamine neurons in the SN and their axons in the striatum, and the TDP-43 specific cell loss, i.e., not seen in controls, was dependent on the vector dose. Consistent with the dopaminergic disease sequelae induced by TDP-43, the rats had a vector dose-related motor activity defect, therefore modeling motor aspects of ALS and FTLD. The histological analysis revealed ubiquitinated aggregates only in the TDP-43 animals, which underscores the mimicry of human TDP-43 proteinoathies which include TDP-43 and ubiquitin lesions. In human TDP-43 diseases, the lesions are cytoplasmic in neurons and glia. TDP-43 accumulated in neuronal cytoplasm and nuclei (its normal compartment) in the rat gene transfer assay. TDP-43 gene transfer induced specific astrogliosis and microgliosis not seen with controls.

While expressing a disease-related gene such as TDP-43 with an efficient vector such as AAV9 is straightforward as of its discovery in neuropathological lesions in 2006, this invention offers advantages for studying TDP-43 and for TDP-43 disease drug development. We have demonstrated a TDP-43 neuropathological effect, i.e., neuronal cytoplasmic accumulations of exogenously applied TDP-43 that co-localizes with the host organism'subiquitin, a TDP-43 neurotoxic effect, i.e., destruction of dopamine neurons, and a functional phenotype, i.e., motor behavior defect. To our knowledge a rodent assay of TDP-43, via viral vector or transgenesis, e.g. transgenic mice, is heretofore unreported, although a yeast model has recently surfaced (28). While knockout mice for superoxide dismutase achieve motor neuron degeneration similar to ALS, they lack the main neuropathology of ALS, cytoplasmic TDP-43 lesions (23).

### Hippocampal Administration

Hippocampal sclerosis is often found in TDP-43 diseases including ALS, and could be related to the cognitive decline and dementia in FTLD. Disclosed herein are methods for expressing TDP-43 in the hippocampus. The assays provided thereby can be evaluated for neuropathology and memory effects, specifically comparing the induced disease state when TDP-43 expression is targeted to the nucleus or the cytoplasm.

Disclosed herein are methods for inducing TDP-43 pathology in either the hippocampus or the hypoglossal nucleus by injecting AAV9 TDP-43 into those regions. TDP-43 pathology in the hippocampus is relevant to FTLD-TDP, AD, and hippocampal sclerosis (Dickson et al., 1994; Josephs et al., 2009; Josephs & Dickson, 2007; Amador-Ortiz, 2007; 2007a), as well as ALS (Neumann et al., 2009). TDP-43 pathology in the hypoglossal nucleus is relevant to FTLD-TDP (Josephs et al., 2009) and to ALS (Eisen, 2009). Bulbar onset ALS makes up 25% of ALS cases, typically has a worse prognosis than spinal onset ALS, and often first manifests as dysarthria, or orolingual dysfunction disrupting normal speech and eating, due to degeneration of the motor neurons that control the tongue in the hypoglossal nucleus in the brainstem (Tomik & Guiloff, 2008; DePaul et al., 1988). Dysarthria is therefore an important sign of pathogenesis. The methods disclosed herein express TDP-43 in either the hippocampus or the hypoglossal nucleus and thus provide assays to study functional effects relevant to hippocampal sclerosis or dysarthria. A key point in TDP-43 diseases, such as ALS (Eisen, 2009) is that disease pathogenesis occurs before symptoms are present, so it could be possible to study early pre-symptomatic changes in the gene transfer assay disclosed herein.

TDP-43 pathology in FTLD involves aberrant deposition in the cytoplasm, therefore, it is contemplated that directing expression to the cytoplasm may result in more disease-like pathology. However, it may be that nuclear, rather than cytoplasmic, overexpression is more deadly to hippocampal neurons, and therefore more likely to affect behavior. A TDP-43 cleavage product of 25 kDa is often found in disease samples, and when this fragment was expressed in cultured cells, it had the toxic function of recruiting nuclear TDP-43 to the cytoplasm. It is therefore contemplated that TDP-25 may be more prone to aggregation, phosphorylation, and ubiquitination relative to wild type TDP-43, and clear endogenous rat TDP-43 from the nucleus. It is further contemplated that there will be greater susceptibility to TDP-43 induced neurodegeneration in aged subjects because neurodegenerative diseases, including TDP-43 diseases, are age related. It may therefore be that aged models will be more sensitive to low level TDP-43 expression and provide greater amplitude effects, mimicking the human age dependence.

### Intravenous Administration

To model the widespread CNS TDP-43 pathology pattern of FTLD and ALS, methods for widespread CNS TDP-43 gene transfer via intravenous vector administration are provided. It has been suggested that AAV9 has unique properties for CNS transduction after systemic delivery, and therefore provides a method for mimicking the widespread brain and spinal cord pathology in FTLD and ALS. It is contemplated that systemic AAV9 TDP-43 will impair motor function and cause frank degeneration of upper and lower motoneurons and their axons, when TDP-43 expression is directed to either the nucleus or the cytoplasm. Neurodegeneration of the hippocampus and hypoglossal nucleus can thus be compared to the results from the intraparenchymal injections above. The TDP-43 induced disease state assay can be confirmed to mimic the muscular degeneration of ALS by longitudinal electromyography and postmortem muscle histology. Dose-response will mimic either rapid progression to end stage, or a more slowly progressing disease state.

Provided herein are methods to transduce the spinal cord and brain globally by injecting rats intravenously with TDP-43 and a suitable vector. In some embodiments, the vector is AAV9. Foust et al. (2009) used AAV9 green fluorescent protein (GFP) in one day old mice and found that 60% of spinal cord neurons were positive for GFP 10-21 days post-injection. Duque et al. (2008) also achieved significant spinal cord transduction applying AAV9 intravenously to adult mice. The assays provided by the intravenous gene transfer methods disclosed herein can be used for assaying FTLD and ALS as TDP-43 pathology in the CNS is widespread in FTLD and ALS (Geser et al., 2008; 2009).

In order to drive TDP-43 to the cytoplasm, a vector for TDP-43 was generated with a mutation in the nuclear localization signal (NLS), to better mimic cytoplasmic TDP-43 pathology. Using the methods disclosed herein, a vector for a 25 kDa disease-related C-terminal TDP-43 fragment can also be provided. This fragment is considered to be one of the major pathological species in TDP-43 diseases (Zhang et al., 2008; 2009; Rohn, 2008; Nonaka et al, 2009; Caccamo et al., 2009). These TDP-43 variants will improve the degree of cytoplasmic TDP-43 accumulation and allow determination of in vivo toxicity.

It is contemplated that many other known infusion methods could be applied including convection-enhanced delivery, intracerebroventricular and intrathecal infusions, pulled glass pipet injection cannulae, and other infusion parameters and strategies known in the field.

Provided herein is a neurodegenerative disease assay of spinal onset ALS based on TDP-43. In some embodiments, the assay is a rodent, such as a rat or a mouse. It is contemplated that: 1) widespread TDP-43 gene transfer can be achieved in the spinal cord and therefore provide a behaviorally and neuropathologically relevant assay of ALS; and 2) TDP-43 variants designed to express in the cytoplasm are more likely to produce a human-like pattern of cellular pathology, but less likely to produce a behavioral phenotype relative to wild type TDP-43. The methods disclosed herein can provide an assay for widespread CNS transduction in neonatal and adult rodents, depending on the vector dose employed. The model of ALS disclosed herein would encompass widespread TDP-43 pathology in the spinal cord and brain (Geser et al., 2008) along with ALS-like motoric deficits. In some embodiments, the assay is a neonatal rat.

### Coexpression Models

TDP-43 could be coexpressed with other modulating transgenes. Viral vectors could be combined and applied to the rat simultaneously, and we successfully expressed more than one vector (tau and parkin (29); tau and GFP (25)). Alternatively, a viral vector could be applied to a transgenic mouse line to study transgene combinations, a strategy with which we have had success (tau or GFP AAV in amyloid transgenic mice (30)). Strategies to exacerbate a TDP-43 disease phenotype in laboratory subjects could include coexpression with ALS-related mutant SOD, or to comprehensively mimic combined neuropathologies including TDP-43 neuronal cytoplasmic inclusions (good to define) and neuronal intranuclear inclusions, along with Lewy bodies or neurofibrillary tangles, with either alpha-synuclein or tau coexpression, respectively.

On the other hand, many neuroprotective factors are hypothesized: an anti-apoptotic factor such as XIAP because TDP-43 can induce apoptosis; a neurotrophic factor such as GDNF; the gene parkin which could ubquitinate and degrade TDP-43; the gene CMP2B which could lead TDP-43 to endosomal degradation. The coexpression could occur by simultaneous injection or by serial injections. For serial injections, the serotype of the vector could be changed to avoid any immune-related neurtralization of transgene expression using the same serotype in succession.

### Formulations

The present invention includes formulation for the introduction of the expressible gene construct into a non-human animal. In one embodiment, the formulation comprises an aqueous buffered solution which comprises an expressible gene construct comprising the TDP-43 gene, wherein the formulation is suitable for injection, infusion and/or intravenous delivery into a non-human animal. Such aqueous solutions are well known in the art and may include by way of example, electrolytes such as NaCl, KCl, CaCl₂, and sodium lactate, alcohols and/or polyols such as mannitol, sugars such as dextrose, amino acids, or solutions comprising multiple electrolytes and mixtures thereof. In some embodiments, the formulation comprises a solution such as ringer's solution or lactated ringer's solution.

### Analysis of Efficacy

Once an assay for a proteinopathy has been generated in rodents using TDP-43, the effects of other interacting gene products or small molecules can be evaluated by either histological analysis of brain sections, metabolic analysis in live animals or behavioral analysis. Histological analysis is carried out by immunohistological staining to look at the morphology of affected cells as well as the protein compartmentalization in regards to TDP-43. The peroxidase immunostaining method allows for the practice of stereology, where eight evenly spaced 50 μm thick sections of the SN of the rat are evaluated for the number of positively stained tyrosine hydroxylase (TH) neurons present. Through the use of MicroBrightfield, Inc. software an estimate of the total number of TH positive neurons in the rat's SN can be generated. After this procedure was performed the results seen in Table 1 were generated for SN TH positive neurons. As can be seen in Table 1 uninjected and GFP control animals had roughly 9500 TH positive neurons per SN. This was decreased in a dose-dependent manner after treating animals with TDP-43 for 4 weeks to approximately 2000 to 5000 cells, depending on the dose of TDP-43 given. The same is true with regard to measures of optical density of TH axons in the striatum: a dose-dependent decrease in the TDP-43 vector groups and no effect from control GFP vectors (Table 1). Table 1 also shows that the TDP-43 induced reductions in dopaminergic markers (TH neurons in the SN and fiber density in the striatum) can be successfully studied in aged rats as well as young.

TABLE 1
TDP-43 gene transfer causes dopaminergic neurodegeneration

| | Dose | | TH fiber density | |
|---|---|---|---|---|
| Vector | (×10¹⁰ vg) | TH neurons | (% uninjected side) | N |
| Uninjected | — | 9470 ± 357 | — | 6 |
| GFP | 1.0 | 9337 ± 440 | 98 ± 5 | 8 |
| GFP | 3.0 | 9609 ± 366 | 101 ± 3 | 6 |
| TDP | 1.0 | 4605 ± 836* | 52 ± 10* | 8 |
| TDP | 3.0 | 1858 ± 167*,** | 6 ± 3*,** | 6 |

Interval of 4 weeks for the vector groups.
*different than dose-matched GFP counterpart, P < 0.001, ANOVA/Bonferroni.
**TDP-43 vector dose differences for TH neurons (P < 0.01) and fiber density (P < 0.001) in ANOVA/Bonferroni.

While the above analysis provides a means of quantifying cell numbers and specific protein expression, neuropathies are often manifested by changes in brain activities. Changes in brain activity or function can be analyzed both in TDP-43 transgenic animals, as a baseline in determining changes induced by this protein, and in compound treated TDP-43 transgenic animals or in TDP-43 transgenic animals in which two or more additional genes are introduced, through use of standard functional assays which are well known to those skilled in the art and include, PET, SPECT, MRI, and biophotonic imaging. An increase in metabolic activity following the addition of a small molecule indicates a potential therapeutic. In the context of assaying in the nigrostriatal system, neurochemicals including dopamine and its metabolites would be measured. Votammetry and electrophysiology could also monitor both effects of TDP-43 and the potential for protective blocking effects of coexpressed genes. When an interacting gene product(s) is analyzed, either on increase or decrease in brain function could lead to a better understanding of the mechanisms associated with this disease, which in turn could result in better assays for analyzing small molecule effects on the disease.

Additionally, the effects of small molecules or interacting gene products can be analyzed in behavioral assays of TDP-43 proteinopathy related to FTLD-U or ALS. Examples of such behavioral assays include amphetamine-stimulated rotational behavior, the rotarod test, limb use asymmetry (cylinder test), the vertical pole test, open field for overall locomotion, balance beam, elevated plus maze, learned helplessness, Morris water task, passive avoidance, radial arm maze, alternating Y maze, forepaw reaching test, forepaw adjustments steps, active place avoidance, contextual fear conditioning, avoidance fear conditioning, inhibitory avoidance, cocaine conditioned place preference, conditional taste aversion, taste familiarity, motor memory, and skilled forepaw reaching task. An improvement in behavioral performance following treatment with a small molecule indicates a potential therapeutic.

In the present invention, histochemistry was used to demonstrate similar pathology and cell loss in prefrontal lobe neuropathies. The above analyses can therefore be used with this invention to demonstrate in vivo efficacy of small molecules or effects of TDP-43 interacting proteins on this assay of proteinopathy resulting in still better assays of the diseases or in new targets for screening of potential therapies.

### Therapy

A compound identified by any of the above-described methods may be administered within a pharmaceutically acceptable diluent, carrier, or excipient, in unit dosage form. Conventional pharmaceutical practice may be employed to provide suitable formulations or compositions to administer the identified compound to patients suffering from a proliferative disease. Administration may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (19th ed., A. R. Gennaro, ed., 1995, Mack Publishing Company, Easton, Pa.). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds that modulate TDP-43 function or levels include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound identified according to the methods described above, may be combined with more traditional therapies for a proliferative disease, for example, traditional chemotherapeutic agents, radiation therapy, or surgery. In addition, these methods may be used to treat any subject, including mammals, for example, humans, domestic pets, or livestock.

The criteria for assessing response to therapeutic modalities employing an identified compound are dictated by the specific condition and will generally follow standard medical practices. Generally, the effectiveness of administration of the compound can be assessed by measuring changes in characteristics of the disease condition.

### Gene Therapy

TDP-43 interacting gene products can themselves be used as therapies when such gene products prevent the deficits associated with TDP-43 over expression. One way to achieve this goal is to ‘knock-down’ or ‘silence’ gene expression. Some such examples of technologies that are able to do this include: Oligodeoxynucleotides, siRNA, shRNA, miRNA, and morpholinos. By inhibiting the expression of a gene product that is causing a proteinopathy it is possible that the effects of said proteinopathy could be lessened or completely ameliorated. Our data suggest that TDP-43 neurotoxicity is related to vector dose and expression levels, so manners in which TDP-43 expression could be blocked would be of value. The longterm goal would be to develop an enzyme factor that targets TDP-43 specifically for degradation, that acts specifically on pathological TDP-43, the hyperphosphorylated cytoplasmic TDP-43, and not normal TDP-43 or a host of other gene products. If such a specific factor could found by gene coexpression studies, perhaps a rational drug design strategy could be adopted to find a way to modulate the normal enzyme in situ with a small molecule drug. On the other hand, the drugs to treat ALS and FTLD-U are sadly lacking and time from diagnostic symptoms to death is relatively short, so new therapies are worth considering. The familial mutations that cause FTLD-U and ALS, (progranulin, TDP-43, SOD, CHMP2B, Valosin containing protein) also make a corrective gene therapy worth considering. The exploding new field of TDP-43 neurobiology in neurodegenerative diseases is not without rational gene targets for coexpression experiments, such as an anti-apoptotic factor, or enzymes that could specifically target pathological TDP-43 for ubiquitination and degradation such as parkin or CHMP2B.

### EXAMPLES

### Example 1

### Rat TDP-43 Assay

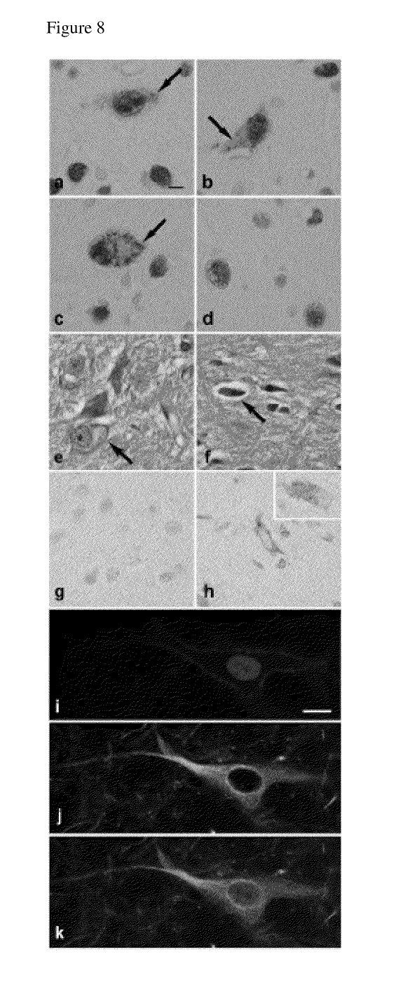

In order to expand FTLD modeling to a major fraction (50%) of the disease population, TDP-43 in was expressed rats, and a highly specific toxic gene product was observed, causing neuronal cell loss more readily than tau. The human wild type TDP-43 mainly expressed in neuronal nuclei as expected, but a small fraction of the transduced cells (estimated to be 1-2% of the total) expressed TDP-43 in the cytoplasm. Three such examples are shown at high power in FIG. 8A-C, with one example from the GFP group in FIG. 8D, using an antibody that recognizes both rat and human TDP-43. TDP-43 positive nuclei are in both groups, whereas the protein is only expressed cytoplasmically in the TDP-43 group, with diffuse and granular immunoreactivity at an interval of 4 weeks. Cytoplasmic ubiquitin deposition only occurred in the TDP-43 group, in the areas that had the cytoplasmic TDP-43 (FIG. 8H). There was evidence of abiotrophic, pyknotic degeneration by hematoxylin & eosin staining (FIG. 8F) and apoptosis in the TDP-43 injected areas. At high power with a confocal microscope, all of the cells overexpres sing TDP-43 (using an antibody specific for human TDP-43) had immunoreactivity dotted along the plasma membrane (FIG. 8I-K). It is contemplated that the plasmalemmal TDP-43 staining can be co-localized with several markers by confocal, and also with the RNA stress granule marker staufen in the cytoplasm.

Many functions have been attributed to TDP-43 related to nuclear DNA (Buratti and Baralle, 2008), however, TDP-43 functions outside of the nucleus should be investigated as well, e.g. functions relating to stress and injury response and synaptic plasticity (Wang et al., 2008; Moisse et al., 2009; 2009a). The AAV9 TDP-43 can therefore be titrated to achieve vector dose-dependent effects on loss of dopamine neurons and their axons in the striatum, as well as amphetamine-induced rotational behavior.

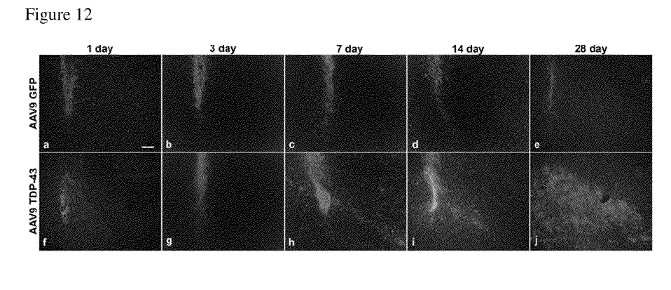

Microgliosis specific to TDP-43 was observed that increased from 1-28 days (FIG. 12), and was vector dose-dependent, measured by a semi-quantitaive optical density method of Cd11b immunoreactivity. The microglial staining was estimated to be elevated 100-fold relative to the contralateral side in groups of 5-6 rats, while the matching GFP tissues had 1.4 fold elevation at 28 days relative to the non-transduced side. DAPI counterstaining matched with the microglial staining with a dramatic cellular infiltration, while staining for the neuronal marker NeuN showed widespread loss of cells in the SN and, in the high dose group, outside the substantia nigra, so the overall cellular effects after TDP-43 gene transfer were more robust and dynamic than was observed with any of the other genes tested.

### Example 2

### TDP-43 Expression in the Hypoglossal Nucleus

a) An assay which mimics symptoms of bulbar onset ALS can be provided using the methods for TDP-43 gene transfer to the hypoglossal nucleus disclosed herein. It is contemplated that focal delivery of TDP-43 to the rat hypoglossal nucleus will mimic a specific symptom of bulbar onset ALS (Eisen, 2009; DePaul et al., 1988) as described herein. Bulbar onset ALS with dysarthria has a particularly poor prognosis due to aspiration of food (Tomik and Guiloff, 2008). Dysarthria, or motor dysfunction of the mouth, is a key symptom of bulbar onset ALS, where the face, and talking, are affected (DePaul et al., 1988). The hypoglossal injections of AAV9 TDP-43 into rats induces a robust phenotype of decreased lick force, reminiscent of dysarthria.

The methods disclosed below test whether TDP-43 variants (NLS TDP, TDP-25) designed for cytoplasmic expression will also impair lick force. It is contemplated that while TDP-43 expression directed to the cytoplasm will lead to more disease-like neuropathology, they will not be as toxic to hypoglossal neurons, and therefore not cause such a pronounced behavioral effect. It is further contemplated that the full length TDP-43 will be the most toxic, presuming that it disrupts normal nuclear function, while the cytoplasmic forms will produce more neuropathology that faithfully mimics human disease, although they will be less likely to cause neuronal loss in the brainstem. Using the methods disclosed herein coupled with what is known in the art, the AAV9 vector for NLS TDP and the AAV9 vector for TDP-25 can be prepared. The vectors can then be stereotaxically injected (5×10E9 vg per side) into the hypoglossal nuclei on each side of 300 g S.D. male rats, that were previously trained and measured in the lick force test.

Lick force test: Because the lick force test uses pre-treatment baseline behavior, the GFP group shall suffice as control for behavior, but six vehicle injected rats can serve as controls for the histological assessments. The lick force tests can run before injections, and 1, 2, 4, and 8 weeks after with each session lasting two minutes using rats who have been maintained for water intake (Vertebrate Animals). The lick waveforms can be analyzed for frequency and force (expressed in Hz and g, respectively) and be compared over time by repeated-measures ANOVA with specific intergroup differences by Bonferroni multiple comparisons.

Histology: TDP-43 expression can be evaluated in the nucleus and cytoplasm with the human specific antibody, and stereological assessments can be made of ubiquitin expression and phospho-TDP-43. It is contemplated that more cytoplasmic expression of TDP-43 will be observed as well as ubiquitination and phosphorylation with the two variants relative to wild type, and therefore better mimicry of the neuropathology of FTLD and ALS can be provided. It can also be confirmed that TDP-25 can clear endogenous TDP-43 from the nucleus using the methods disclosed herein, as is shown in vitro (Nonaka et al., 2009; Caccamo et al., 2009). The neuronal profiles in the hypoglossal nucleus can be quantified by stereology. The motoneurons stained for choline acetyltransferase (ChAT) could be counted, although stereology for NeuN would also suffice to evaluate all of the neurons in the hypoglossal nucleus, a relatively small structure. Pathological assessments of gliosis and apoptosis can be performed as above. The measures of pathology and cell loss can then be correlated to the lick force data. The full length TDP-43 is predicted to be the most robust for decreased lick force and neuronal loss, although a behavioral phenotype with one of the other two variants could result in a better human model because the cellular neuropathology will be more faithful to the human pattern. If behavioral and neurodegenerative effects are only found in the full length wild type TDP-43 group, it would suggest that disruption of normal nuclear functions are more likely to underlie disease, rather than the cytoplasmic expression which could be bystander.

It may be that the cytoplasmic variants are less toxic than wild type and thus require higher vector doses to affect behavior. However, with the total dose of 1×10E10 vg injected, some lick force effects are expected with the cytoplasmic forms by 8 weeks, which occurs as early as 10 days with wild type TDP-43. It is further contemplated that greater orolingual deficits in the wild type TDP-43 group will be observed, as well as greater cytoplasmic TDP-43 deposition, ubiquitination, and phosphorylation with the two variants relative to wild type, and lick force deficits that correlates with degree of pathology and cell loss. Should little or even an inverse correlation result between ubiquitinated TDP-43 deposits and lick weakness, the concept of the pathology as a protective response could be considered.

b) TDP-43 has been expressed in regions relevant to ALS such as the hypoglossal nucleus in the brain stem. The hypoglossal nuclei are delineated with GFP expression. Orolingual function is studied after TDP-43 gene transfer to this nucleus. There appears to be a striking debilitation induced specifically by TDP-43. The licking behavior is measured by an isometric strain gauge. Water deprived rats lick the bar to receive a water drop reward. Dysarthria is mimicked in affected TDP-43 rats. A dose effect is observed, as well as the time-dependence from pre-treatment behavior up to 3 weeks after, with apparent deficits as early as 6 days at the high dose.

### Example 3

### Intravenous Administration of TDP-43 AAV9

It is contemplated that widespread TDP-43 expression from intravenous vector delivery is relevant to diseases with widespread pathology. A method for gene transfer to the spinal cord, brain stem, and brain via intravenous vector administration is thus provided.

a) One day old rat pups were injected with GFP or TDP-43 AAV9 via the temporal vein with a 30 ga. needle. A high dose of AAV9 TDP-43 in neonatal rats caused a severe and rapid disease state as early as 3 weeks. Therefore, a low dose (9×10E11 vg) should permit a slower pathogenesis. Transduced rats can be maintained for 6 months for evaluating their behavior, as well as overall health and weight gain over 6 months. Behavioral testing intervals can be at 1, 2, 3, and 6 months before histological analysis at that interval, with 12 rats per group. Each vector group can have 6 rats sacrificed at 1 month for histology, which would not be run for behavior.

A dose-response can be evaluated for the full length wild type TDP-43, but lower or higher doses of the NLS TDP or TDP-25 could be incorporated if induced disease states are quite robust or absent, respectively. Male and/or female rats can be used.

Behavioral tests: The rats can be monitored for locomotor activity in open field testing using a photobeam activity monitoring system (TruScan, Coulbourn), and rotorod (Rota-rod, Stoelting) performance. The total distance traveled can be compared and number of rearings in the open field in 30 min sessions, and times to fall off an accelerating rotorod (from 4 to 40 RPM over 2 min) as gross measures of motor function. For the rotorod, data are collected by running 3 trials per day after an initial training session. Total forelimb usage can be incorporated in the cylinder test, skilled forelimb use with a pellet baited staircase apparatus, or ability to husk and eat unsalted sunflower seeds, the latter two tests with fasted animals (Kleim et al., 2007). Other tests can be performed, such as lick force, grip strength, gait analysis, ability to hang and suspend on a string, and righting reflex. Behaviors can then be compared over time by repeated measures ANOVA and Bonferroni multiple comparisons for normally distributed data or non-parametric tests (e.g., sunflower seed test), as appropriate.

Histology: Spinal cord and brain as well as other tissues can be harvested for expression analysis. If GFP fluorescence is weak, immunostaining can be performed, as well as PCR based detection. The spinal cord can be systematically analyzed in blocks at various levels in cross-section, as well as the brain stem, and brain to evaluate transgene expression, neuronal loss, and TDP-43-specific atrophy. Motoneurons stained with ChAT or another commercially available marker, such as SMI32 can also be assessed. Watson et al. provides high resolution images for several markers (ChAT, AChE, NeuN, Nissl, among others), which allows for stereological estimates of defined sections of the cord (e.g., the cervical and lumbar enlargements). Neuronal and glial counterstains can also be run, and it is expected to find expression in glia by this gene delivery method (Foust et al., 2009; Duque et al., 2009), potentially relevant to human glial TDP-43 pathology (Nishihira et al., 2008). The fraction of spinal cord motoneurons expressing GFP can be evaluated. Spinal sections can be processed for toluidine blue to assess axonal degeneration at all levels of the cord (i.e., number and thickness of axons), and apoptosis. Gastrocnemius muscle tissue can be processed for hematoxylin and eosin staining which is predicted to show cellular signs of atrophy and wasting due to the axonal loss and dennervation (Biosketch and Letter of Support; Bice and Beal, 1997; 1997a; Nandi et al., 1993). Animal weights, organ tissue weights, neuronal numbers and sizes, and volumes of selected neuroanatomical structures can be compared by ANOVA/Bonferroni multiple comparisons across groups. TDP-43 transduction and cellular effects can then be evaluating in the hippocampus and hypoglossal nucleus by comparing the results from intravenous and intraparenchymal gene delivery.

Other non-CNS assessments: Electromyography of the gastrocnemius muscles can quantify the presence of fasiculations, an ALS relevant symptom. Fasiculations, the involuntary spasms which are a sign of dennervation, are likely to occur during the early stage in progressive dennervation induced by TDP-43, when the hindlimbs become spastic, but not at end stage when the dennervation is more complete and the muscles are flaccid. Specific effects on the nervous system can be demonstrated, so various organs (liver, kidney, heart, lung) can be weighed upon necropsy to evaluate whether there is specific atrophy of the brain, spinal cord, and muscle relative to other organs. It is contemplated that analysis of liver enzyme levels or function would be a good control for neuromuscular specificity. PCR analysis of vector genomes in various tissues can provide a vector spread throughout the body.

Sufficient expression levels to achieve a TDP-43 specific behavioral effect is observed, based on the rats disclosed herein. The doses for intravenous delivery may be too potent and titration to even lower doses can be performed if the TDP-43 rats are too rapidly debilitated. If such is the case, the 6 month interval could be truncated. Alternatively, if there is insufficient expression, the woodchuck hepatitis post-transcriptional element (WPRE) can be used to boost levels, or one could adopt double stranded self complementary DNA AAV technology as used by McCarty et al. (in press). Other alternatives could be used to compare AAV9 to another serotype, or to express familial FTLD TDP-43 mutants (Gitcho et al., 2008; Sreedharan et al., 2008). Intact spinal cords of young and adult rats have been successfully harvested using the methods disclosed herein.

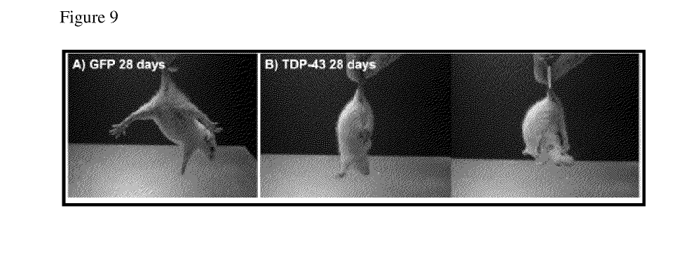

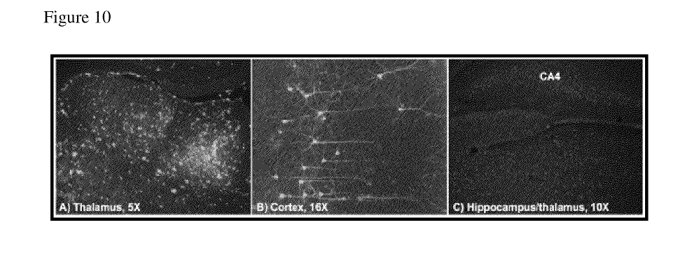

b) AAV9 TDP-43 vector has been applied to adult rats. Trends for motoric impairments were observed for the TDP-43 rats relative to GFP over 6 weeks, but preliminary results from neonatal injections were far more striking. Rat pups were injected in the manner done with mice in Foust et al. (2008; 2009) with a dose of 9.0×10E11 or 1.5×10E12 vg. The motor phenotype was severe in the high dose (h.d.) TDP-43 rat within 3-4 weeks. While normal rats, and a GFP rat (N=1), extend their hindlimbs when picked up by their tail (an escape response), the h.d. TDP-43 rat (N=1) crossed its legs, and showed signs of drastic loss of gastrocnemius muscle tone, indicative of end stage dennervation (FIG. 9). Rotorod and open field behavior are sufficient to quantify the deficits. The GFP rat appeared normal for ambulation on a rotorod compared to untreated controls, but the h.d. TDP-43 rat could not walk on the rotorod at all as early as 3 weeks, even at the slowest RPM. In the open field, the h.d. TDP-43 rat propelled itself on its abdomen and only with its forelimbs, leading to marked differences for distance traveled and rearing relative to controls. The h.d. TDP-43 rat showed impaired weight gain relative to GFP rats and gastrocnemius muscle wasting upon necropsy at 32 days. Examples of transgene expression are in FIGS. 10 & 11, which confirm specific GFP or TDP-43 in the respective groups.

The loss of hindlimb gait and function was similar to mutant TDP-43 transgenic mice, although it occurred sooner in the vector-based assay (3 weeks vs. 5 months) and from wild type TDP-43. Low TDP-43 vector dose can be used to achieve a progressive disease that does not too rapidly impair the animal, and therefore prevent it to be analyzed over time, in order to study a range of disease stages. An advantage of the vector system is that transgene expression levels can be controlled. ALS is a disease of older adults, and the pathogenesis begins before symptoms (Eisen, 2009). Using the methods and assays disclosed herein, one could study both early and later stages with dose-response and time-course.

### Example 4

### Modulation the TDP-43 Induced Disease State with Progranulin co-Expression

To determine if the TDP-43 induced disease state is treatable, progranulin can be co-expressed, which is contemplated to be protective. However, there are many possible genes to target, such as survival motor neuron (Passini et al., 2009), or trophic factors IGF-1 or GDNF (Kaspar et al., 2003). Mutations in progranulin (presumed to be loss of function) ultimately lead to TDP-43 pathology in FTLD (Baker et al., 2006; Leverenz et al., 2007; Shankaran et al., 2008), and progranulin overexpression appears to be protective in a preliminary zebrafish TDP-43 model (Van Damme, 2009), so it is therefore a rational target for a rodent assay of TDP-43 diseases. Because loss of progranulin function could underlie these familial forms, progranulin gene delivery could potentially restore function. Behavioral and cellular protection of progranulin co-expression can be assessed in the intravenous AAV9 assay. Should progranulin prove ineffective, neurotrophic factor gene delivery can be proof that the TDP-43 induced disease state is amenable to therapy.

cDNA for human progranulin is commercially available (which is not too large to fit into AAV) from Open Biosystems, and an AAV9 vector is generated. Detection of progranulin expression can rely on antibodies, or PCR if necessary. The intravenous delivery assay to neonatal rats can be used to assess motor function and neuromuscular degeneration induced by TDP-43 expression. Rats can receive matching equal doses of TDP-43/Empty vector or TDP-43/Progranulin or Progranulin/Empty totaling 1.8×10E12 vg.

A full length TDP-43 can be used so that an accumulation of fragments in the cytoplasm might be observed, which is potentially due to processing of TDP-43 by progranulin by an enzyme cascade. Progranulin was reported to mediate caspase dependent cleavage of TDP-43 in vitro (Zhang et al., 2008), although there was a conflicting study (Dormann et al., 2009). If able to lessen the behavioral and cellular degeneration induced by TDP-43 with progranulin, it is contemplated that the assays disclosed herein can be used to pursue biochemical studies which address caspase dependence.

If difficulty detecting progranulin expression in pilot rats is observed, increasing the dose or adding WPRE can be performed. TDP-43 can be simultaneaously co-expressed with progranulin, although administering progranulin after TDP-43 can be performed as well in the methods disclosed herein. The methods disclosed herein can also be used with progranulin to study two proteins interacting either directly or indirectly in enzyme cascades in the cytoplasm. GDNF is an effective survival factor for spinal motor neurons in a familial SOD transgenic mice assay (Kaspar et al., 2003), and a behaviorally confirmed GDNF vector (Klein et al., 2005) could be applied.

### Example 5

### Protocols

Methods sections for vector production, injections, dissections, histology, stereology, Morris water task or locomotor or rotorod behavior, and the statistics have been published (Klein et al., 1999; 2000; 2002; 2008; 2008a; 2009; Tatom et al., 2009). The protocol for orolingual function is in Smittkamp et al., 2008. Histological analyses for the spinal cord will follow Bice and Beal, 1997; 1997a; Foust et al., 2009.

DNAs: The expression cassette is flanked by the AAV2 terminal repeats, and contains the hybrid CMV/chicken beta actin promoter, and the bovine growth hormone polyadenylation sequence. Plasmids are propagated in SURE cells (Stratagene) and purified by CsCl ultracentrifugation.

Vector packaging and titering: Plasmids are packaged into recombinant AAV9 by a three plasmid system (two helper plasmids). Both helper plasmids were provided by the Gene Therapy Program, University of Pennsylvania, Philadelphia, Pa. Preparations are titered for DNAse-resistant vector genomes (copies) by non-radioactive dot-blotting using the Brightstar kit (Ambion).

Subjects and gene delivery: Male Sprague-Dawley rats (either 3 or 20 months old) are anesthetized with cocktail made up of 3 ml xylazine (20 mg/ml), 3 ml ketamine (100 mg/ml), and 1 ml acepromazine (10 mg/ml) administered intramuscularly at a dose of 0.5-0.7 ml/kg. The injection coordinates derive from Paxinos & Watson (1998). Virus stocks are injected through a 27 ga. cannula connected via 26 ga. I.D. polyethylene tubing to a 10 μl Hamilton syringe mounted to a CMA/100 microinjection pump. The pump rate is 0.25 μl/min. The needle remains in place at the injection site for 5 additional min and then is removed slowly. For intravenous injections, a 30 ga. needle is used to deliver volumes up to 0.1 ml to the temporal vein of one day old rats. A large standing magnifying glass is used. Anesthesia is achieved by wrapping the rat in a latex glove and placing it in an ice bucket for 5 min. The collaborator Elysse Orchard will assist with intravenous injections (Letter of Support). All animal care and procedures are in accordance with our institutional Animal Care and Use Committee and NIH guidelines (Guide for the Care and Use of Laboratory Animals, DHHS Publication No. NIH-85-23).

Western blots: Brain tissue is dissected using a 1 mm brain block and 3 mm dia. circular biopsy punchers for consistent dissections (−17 mg wet weight). The samples are Dounce homogenized in solubilization buffer, centrifuged once at top speed in a microcentrifuge for 5 min, and the supernatant analyzed. Samples are normalized for protein content by Bradford assay and subjected to 12% SDS polyacrylamide gel electrophoresis. Beta-actin immunoblots of the same samples are also used for normalization. Bands on immunoblots are compared using the Scion Image software.

Histological techniques: Primary antibodies include human specific TDP-43 (Abnova), non-species specific TDP-43 (ProteinTech), phosho-TDP (Cosmo), ubiquitin (Dako), NeuN (Chemicon), ChAT (Chemicon), Cd11b (Chemicon), GFAP (Chemicon), staufen (Abnova), GFP (Invitrogen), among other markers of motoneurons and plasma membrane as necessary. Counterstains will include DAPI, hematoxylin & eosin, Nissl substance, toluidine blue. Nickel enhanced peroxidase as well as fluorescent staining will be used as necessary, e.g., stereology and confocal, respectively. Stereology requires thick sections (50 microns). The neuropathological assessments can be made on paraffin sections (˜5 microns). Harvesting the spinal cord entails laminectomy going down the dorsal vertebrae with a Rongeurs and scalpel, with standard paraformaldehyde perfused tissue. The entire cord can be sampled and analyzed. Blocks of the cord can be systematically sampled, cross-sectioned and used for ChAT stereology and toluidine blue. Staining for apoptotic cells uses a diamonobenzidine chromagen (Calbiochem). Secondary and conjugated antibodies are from Jackson, Invitrogen, or Sigma. Zeiss microscopes (Axiovert 200, Axioskop 40) and Axiocam camera are used to capture images, and final figures are compiled using Adobe Photoshop. Confocal imaging uses a Leica TCSFP5 microscope. Muscle tissue will be prepared and sectioned using appropriate methods (paraffin embedding) and analyzed for cellular atrophy and wasting by hematoxylin & eosin.

Stereological estimates: Specific neuronal population numbers are estimated by an unbiased method called the optical fractionator using a microscope with a motorized stage. Cavalieri volume estimates also use the stereology software and motorized stage (from MicroBrightfield).

Morris task: Rats swim in a pool of white colored water and learn the location of a rest platform that is below the surface, presumably involving set cues within the room. The trial is capped at 60 sec if the rat does not “escape” to the platform. Four trials per day are run for 5 days in a row, which in normal rats, is sufficient to demonstrate a learning of the location over time. The platform can be removed to evaluate retention of the spatial location using the camera and tracking software (San Diego Instruments) to measure time to location, location crossings, and time spent in location zone, as searching strategy (vs. random).

Lick-force behavior: Thirsty rats are trained to press a bar for a droplet of water. The bar is an isometric strain gauge. The number of licks and the force in g can be measured. After training, sessions are for 2 min, and the lick waveform data are analyzed for lick frequency and average lick force.

Total locomotor behaviors: The rats are acclimated to the behavior room for 1 hr, then placed in a photobeam activity monitoring system in a dark room. The plexiglass enclosures are 18″×18″ 12″ and have 2 rings of photobeams (Coulbourn Instruments). Sessions are for 30 min and the main readout is distance travelled although more measurements are made (move time, rest time etc.).

Rotorod: After one training day attempting to get the rat to walk on the wheel, sessions are for up to 2 min on an accelerating wheel (from 4-40 RPM over 2 min). Three trials are run per day over 4 days, and this can be repeated monthly. Data are expressed as fall latency (sec).

Statistics: ANOVA, or repeated-measures ANOVA, for main effects, with Bonferroni's post tests to correct for multiple group comparisons will be used. A non-parametric test will be used when appropriate. Prism and StatView software are used for graphing and statistics.

### Vertebrate Animals

Rats are an appropriate host to study the brain, brainstem, and spinal cord in relation to human neurodegenerative diseases. Rats are an appropriate assay for AAV-mediated gene transfer. The data provided indicates that this mammalian species responds to the AAV vector system, and is therefore appropriate for generating assays of TDP-43 overexpression. The rat is suitable for either highly targeted stereotaxic delivery or widespread intravenous delivery to affect specific parts of the CNS related to disease.

Male Sprague-Dawley albino rats (either 3 months or 20 months of age; Harlan Laboratories) can be used in gene transfer studies (estimate: 150 per year). All animals were maintained in the AAALAC approved Health Center Animal Care Facility under the direction of a veterinarian. All animal care and procedures were in accordance with NIH guidelines (Guide for the Care and Use of Laboratory Animals, DHHS Publication No. NIH-85-23). Rats are maintained in standard cages in rooms maintained at 26° C., 50% relative humidity and a 12/12 hr daily light cycle. Twelve to 15 air changes per hr are produced in the housing facility. All animals are observed at least once a day during feeding (Purina Rat Chow) and removal of waste. If any signs of morbidity are noticed, these animals are identified and a treatment plan is made in consultation with an attending veterinarian. Prior to admission into the animal room, all rats are maintained in quarantined facilities for 7-10 days for observation by an animal technician.

Gene delivery: Rats are anesthetized with cocktail made up of 3 ml xylazine (20 mg/ml), 3 ml ketamine (100 mg/ml), and 1 ml acepromazine (10 mg/ml) administered intramuscularly at a dose of 0.7 ml/kg. The stereotaxic injection coordinates are derived from Paxinos & Watson (1998). Virus stocks are injected through a 27 ga. cannula connected via 26 ga. I.D. polyethylene tubing to a 10 μl Hamilton syringe mounted to a CMA/100 microinjection pump. The pump rate is 0.25 μl/min. The needle remains in place at the injection site for 5 additional min and then is removed slowly. For intravenous injections, a 30 ga. needle is used to deliver volumes up to 0.1 ml to the temporal vein of one day old rats. Anesthesia is achieved by wrapping the rat in a latex glove and placing it in an ice bucket for 5 min. Immediately following surgery, animals are placed on a warm pad. They are monitored until they exhibit full behavioral recovery from the anesthetics, at which time they are returned to their home cage, where they are housed in pairs in the main animal care facility.

The following tasks do not involve any food or water deprivation, and can be run at set intervals (e.g., 1, 2, 3, 6 months) after the gene transfer treatments.

Morris task: Rats swim in a pool of white colored water and learn the location of a rest platform that is below the surface, presumably involving set cues within the room. The trial is capped at 60 sec if the rat does not “escape” to the platform. Four trials per day are run for 5 days in a row, which in normal rats, is sufficient to demonstrate a learning of the location over time.

Total locomotor behaviors: The rats are acclimated to the behavior room for 1 hr, then placed in a photobeam activity monitoring system in a dark room. The plexiglass enclosures are 18″×18″ 12″ and have 2 rings of photobeams (Coulbourn Instruments). Sessions are for 30 min.

Rotorod: After training to get the rat to walk on the wheel, sessions are for up to 2 min on an accelerating wheel (from 4-40 RPM over 2 min). Three trials are run per day over 4 days.

Electromyography (EMG): Anesthetized rats will be studied for standard EMG at set intervals (e.g., monthly), in a similar manner as routinely done to non-anesthetized humans, and using LSUHSC hospital equipment. Ground and recording electrodes are placed in the gastrocnemius muscle to study muscle fasiculations, a sign of dennervation. Specialized monopolar electrodes will be used.

Histological and biochemical preparation: The animal is anesthetized with the same cocktail as for the stereotaxic injections, at a dose of 1 ml/kg. For histology, animals will be perfused with a 4% paraformaldehyde solution, and the heart is exposed using a mid-line incision and then cutting the ribcage along each side of the thoracic cavity. A 16 gauge needle attached to a peristaltic perfusion pump is inserted into the left ventricle and the right atrium is incised while the pump is turned on allowing the animal's system to be perfused with the paraformaldehyde solution. The procedure takes about ten minutes to complete. For biochemical studies, animals are be anesthetized, and then decapitated with guillotine, and the brains are extracted and frozen on crushed dry ice and homogenized later.

Statistics: Six subjects/group are needed for the histological measures, and 6/group for biochemical measures (westerns) when used to achieve significance from the ANOVA/Bonferroni comparisons. The inherent variability associated with behavioral studies necessitates 12 subjects/group for behavior, based on the results from many previous studies tracking groups of rats after AAV gene transfer and monitoring their behaviors over the course of several months, and then comparing by repeated measures ANOVA. For experiments where sufficient preliminary data was had, a power analysis was run which supported the proposed numbers.

Veterinary care: Following survival surgery, the animals are be monitored daily for the first post-operative week, and weekly thereafter. This entails weighing the animals in addition to monitoring for any abnormalities in their appearance. This post-operative monitoring is in addition to the normal daily monitoring when cages are cleaned. The non-steroidal anti-inflammatory drug flunixin meglamine is injected s.c. at a dose of 2 mg/kg to alleviate post-operative pain. If pain (scratching, rubbing, hunched posture) is observed within the first 1-2 days after surgery, flunixin can be administered at the same dose once a day for 2 days. However, no chronic pain/distress is expected from the procedures. If a very sick animal does result from the treatments or from normal aging, euthanasia may be required. In such a case, a lethal dose of anesthetic cocktail can be administered as above.

### REFERENCES

1. Neumann, M., Sampathu, D. M., Kwong, L. K., Truax, A. C., Micsenyi, M. C., Chou, T. T., Bruce, J., Schuck, T., Grossman, M., Clark, C. M., McCluskey, L. F., Miller, B. L., Masliah, E., Mackenzie, I. R., Feldman, H., Feiden, W., Kretzschmar, H. A., Trojanowski, J. Q., and Lee, V. M.-Y. Ubiquitinated TDP-43 in Frontotemporal Lobar Degeneration and Amyotrophic Lateral Sclerosis. Science, 314: 130-133, 2006.
2. Mackenzie, I., Bigio, E H, Ince, P G, Geser, F, Neumann, M, Cairns, N J et al. Pathological TDP-43 distinguishes sporadic amyotrophic lateral sclerosis from amyotrophic lateral sclerosis with SOD1 mutations. Ann Neurol., 61, 2007.
3. Klein, R., Meyer, E. M., Muzyczka, N., King, M. A., Meyers, C. A. Human disease modeling using somatic gene transfer. US Patent Application, 20040205833, 2004.
4. Klein, R., Meyer, E., Muzyczka, N., King, M., Meyers, C. Human disease modeling using somatic gene transfer. US Patent Application, 20020111321, 2002.
5. Klein, R., Meyer, E., Muzyczka, N., King, M., Meyers, C. Human disease modeling using somatic gene transfer. U.S. Pat. No. 7,098,374, 2006.
6. Klein, R., Meyer, E. M., Muzyczka, N., King, M. A., Meyers, C. A. Human Disease Modeling Using Somatic Gene Transfer. US Patent Application, 20070157324, 2007.
7. Ou, S. H. I., Wu, F., Harrich, D., Garcia-Martinez, L. F., Gaynor, R. B. Cloning and Characterization of a Novel Cellular Protein, TDP-43, That Binds to Human Immunodeficiency Virus Type 1 TAR DNA Sequence Motifs. Journal of Virology, 69: 3584-3596, 1995.
8. Gaynor, R. B., Ou, S.-H.I., Wu, F. K. Cellular protein TDP-43 and regulation of HIV-1 gene expression. U.S. Pat. No. 5,688,511, 1997.
9. Buratti, E., Brindisi, A., Pagani, F., and Baralle, F. E. Nuclear Factor TDP-43 Binds to the Polymorphic TG Repeats in CFTR Intron 8 and Causes Skipping of Exon 9: A Functional Link with Disease Penetrance. The American Journal of Human Genetics, 74: 1322-1325, 2004.
10. Buratti, E., Dork, T, Zuccato, E, Pagani, F, Romano, M, Baralle, F E. Nuclear factor TDP-43 and SR proteins promote in vitro and in vivo CFTR exon 9 skipping. EMBO J., 20: 1774-1784, 2001.
11. Arai, T., Hasegawa, M., Akiyama, H., Ikeda, K., Nonaka, T., Mori, H., Mann, D., Tsuchiya, K., Yoshida, M., Hashizume, Y., and Oda, T. TDP-43 is a component of ubiquitin-positive tau-negative inclusions in frontotemporal lobar degeneration and amyotrophic lateral sclerosis. Biochemical and Biophysical Research Communications, 351: 602-611, 2006.
12. Seelaar, H., Jurgen Schelhaas, H., Azmani, A., Kusters, B., Rosso, S., Majoor-Krakauer, D., de Rijik, M. C., Rizzu, P., ten Brummelhuis, M., van Doom, P. A., Kamphorst, W., Willemsen, R., and van Swieten, J. C. TDP-43 pathology in familial frontotemporal dementia and motor neuron disease without Progranulin mutations. Brain, 130: 1375-1385, 2007.
13. Rollinson, S., Snowden, J. S., Neary, D., Morrison, K. E., Mann, D. M. A., and Pickering-Brown, S. M. TDP-43 gene analysis in frontotemporal lobar degeneration. Neuroscience Letters, 419: 1-4, 2007.
14. Hasegawa, M., Arai, T., Akiyama, H., Nonaka, T., Mori, H., Hashimoto, T., Yamazaki, M., and Oyanagi, K. TDP-43 is deposited in the Guam parkinsonism-dementia complex brains. Brain, 130: 1386-1394, 2007.
15. Catalina Amador-Ortiz, W.-L. L., Zeshan Ahmed, David Personett, Peter Davies, Ranjan Duara, Neill R. Graff-Radford, Michael L. Hutton, Dennis W. Dickson, TDP-43 immunoreactivity in hippocampal sclerosis and Alzheimer's disease. Annals of Neurology, 61: 435-445, 2007.
16. Nakashima-Yasuda, H., Uryu, K., Robinson, J., Xie, S. X., Hurtig, H., Duda, J. E., Arnold, S. E., Siderowf, A., Grossman, M., Leverenz, J. B., Woltjer, R., Lopez, O. L., Hamilton, R., Tsuang, D. W., Galasko, D., Masliah, E., Kaye, J., Clark, C. M., Montine, T. J., Lee, V. M., Trojanowski, J. Q. Co-morbidity of TDP-43 proteinopathy in Lewy body related diseases. Acta Neuropathol., 114: 221-229, 2007.
17. Higashi, S., Iseki, E., Yamamoto, R., Minegishi, M., Hino, H., Fujisawa, K., Togo, T., Katsuse, O., Uchikado, H., Furukawa, Y., Kosaka, K., and Arai, H. Concurrence of TDP-43, tau and [alpha]-synuclein pathology in brains of Alzheimer's disease and dementia with Lewy bodies. Brain Research, 1184: 284-294, 2007.
18. Freeman, S. H., Spires-Jones, T., Hyman, B. T., Growdon, J. H., Frosch, M. P. TAR-DNA binding protein 43 in Pick disease. J Neuropathol Exp Neurol., 67: 62-67, 2008.
19. Bateman, R. J., Holtzman, D. M. Methods for Measuring the Metabolism of Neurally Dervied Biomolecules in vivo. US Patent Application, 20080145941, 2008.
20. Rothstein, J. D. TDP-43 in amyotrophic lateral sclerosis: Pathophysiology or patho-babel? Annals of Neurology, 61: 382-384, 2007.
21. Gijselinck, I., Sleegers, K., Engelborghs, S., Robberecht, W., Martin, J.-J., Vandenberghe, R., Sciot, R., Dermaut, B., Goossens, D., van der Zee, J., De Pooter, T., Del-Favero, J., Santens, P., De Jonghe, P., De Deyn, P. P., Van Broeckhoven, C., and Cruts, M. Neuronal inclusion protein TDP-43 has no primary genetic role in FTD and ALS. Neurobiology of Aging, In Press, Corrected Proof.
22. Banks, G., Kuta, A., Isaacs, A., and Fisher, E. TDP-43 is a culprit in human neurodegeneration, and not just an innocent bystander. Mammalian Genome.
23. Robertson, J., Sanelli, T., Xiao, S., Yang, W., Home, P., Hammond, R., Pioro, E. P., and Strong, M. J. Lack of TDP-43 abnormalities in mutant SOD1 transgenic mice shows disparity with ALS. Neuroscience Letters, 420: 128-132, 2007.
24. Leverenz, J. B., Yu, C. E., Montine, T. J., Steinbart, E., Bekris, L. M., Zabetian, C., Kwong, L. K., Lee, V. M.-Y., Schellenberg, G. D., and Bird, T. D. A novel progranulin mutation associated with variable clinical presentation and tau, TDP43 and alpha-synuclein pathology. Brain, 130: 1360-1374, 2007.
25. Klein, R., Dayton, R D, Tatom, J B, Diaczynsky, C G and Salvatore, M F Tau expression levels from various adeno-associated virus vector serotypes produce graded neurodegenerative disease states. Eur. J. Neurosci., 27: 1615-1625, 2008.
26. Gitcho, M., Baloh, R H, Chakraverty, S, Mayo, K, Norton, J B, Levitch, D et al. TDP-43 A315T mutation in familial motor neuron disease. Ann Neurol., 63: 535-538, 2008.
27. Zhang, H., Tan, C.F., Mori, F., Tanji, K., Kakita, A., Takahashi, H., Wakabayashi, K. TDP-43-immunoreactive neuronal and glial inclusions in the neostriatum in amyotrophic lateral sclerosis with and without dementia. Acta Neuropathol., 115: 115-122, 2008.
28. Johnson, B. S., McCaffery, J. M., Lindquist, S., Gitler, A. D. A yeast TDP-43 proteinopathy model: Exploring the molecular determinants of TDP-43 aggregation and cellular toxicity. Proc. Natl. Acad. Sci. U.S.A., 105: 6439-6444, 2008.
29. Klein, R. L., Dayton, R. D., Leidenheimer, N. J., Jansen, K., Golde, T. E., Zweig, R. M. Efficient neuronal gene transfer with AAV8 leads to neurotoxic levels of tau or green fluorescent proteins. Mol. Ther., 13: 517-527, 2006.
30. Klein, R. L., Lin, W.-L., Dickson, D. W., Lewis, J., Hutton, M., Duff, K., Meyer, E. M., King, M. A. Rapid neurofibrillary tangle formation after localized gene transfer of mutated tau. Am. J. Pathol., 164: 347-353, 2004.

### ADDITIONAL REFERENCES

Amador-Ortiz C, Ahmed Z, Zehr C, Dickson D W (2007a) Hippocampal sclerosis dementia differs from hippocampal sclerosis in frontal lobe degeneration. Acta Neuropathol 113: 245-252.
Amador-Ortiz C, Lin W L, Ahmed Z, Personett D, Davies P, Duara R, Graff-Radford N R, Hutton M L, Dickson D W (2007) TDP-43 immunoreactivity in hippocampal sclerosis and Alzheimer's disease. Ann Neurol 61: 435-445.
Arai T, Mackenzie I R, Hasegawa M, Nonoka T, Niizato K, Tsuchiya K, Iritani S, Onaya M, Akiyama H (2009) Phosphorylated TDP-43 in Alzheimer's disease and dementia with Lewy bodies. Acta Neuropathol 117: 125-136.
Baker M, Mackenzie I R, Pickering-Brown S M, Gass J, Rademakers R, Lindholm C, Snowden J, Adamson J, Sadovnick A D, Rollinson S, Cannon A, Dwosh E, Neary D, Melquist S, Richardson A, Dickson D, Berger Z, Eriksen J, Robinson T, Zehr C, Dickey C A, Crook R, McGowan E, Mann D, Boeve B, Feldman H, Hutton M (2006) Mutations in progranulin cause tau-negative frontotemporal dementia linked to chromosome 17. Nature 442: 916-919.
Bice T N, Beal J A (1997) Quantitative and neurogenic analysis of neurons with supraspinal projections in the superficial dorsal horn of the rat lumbar spinal cord. J Comp Neurol 388: 565-574.
Bice T N, Beal J A (1997a) Quantitative and neurogenic analysis of the total population and subpopulations of neurons defined by axon projection in the superficial dorsal horn of the rat lumbar spinal cord. J Comp Neurol 388: 550-564.
Buratti E, Baralle F E (2008) Multiple roles of TDP-43 in gene expression, splicing regulation, and human disease. Front Biosci 13: 867-878.
Caccamo A, Majumder S, Deng J J, Bai Y, Thornton F B, Oddo S (2009) Rapamycin rescues TDP-43 mislocalization and the associated low molecular mass neurofilament instability. J Biol Chem 284: 27416-24.
Cairns N J, Bigio E H, Mackenzie I R, Neumann M, Lee V M, Hatanpaa K J, White CL, III, Schneider J A, Grinberg L T, Halliday G, Duyckaerts C, Lowe J S, Holm I E, Tolnay M, Okamoto K, Yokoo H, Murayama S, Woulfe J, Munoz D G, Dickson D W, Ince P G, Trojanowski J Q, Mann DM (2007) Neuropathologic diagnostic and nosologic criteria for frontotemporal lobar degeneration: consensus of the Consortium for Frontotemporal Lobar Degeneration. Acta Neuropathol 114: 5-22.
Carlson M E, Hsu M, Conboy I M (2008) Imbalance between pSmad3 and Notch induces CDK inhibitors in old muscle stem cells. Nature. 454:528-32.
Cearley C N, Wolfe J H (2006) Transduction characteristics of adeno-associated virus vectors expressing capserotypes 7, 8, 9, and Rh10 in the mouse brain. Mol Ther 13: 528-537.
Cleveland D. (2009) Murine models of TDP-43 expression. Third International Research Workshop on Frontotemporal Dementia in ALS, June 21-25, London, Ontario.
DePaul R, Abbs J H, Caligiuri M, Gracco V L, Brooks B R (1988) Hypoglossal, trigeminal, and facial motoneuron involvement in amyotrophic lateral sclerosis. Neurology 38: 281-3.
Dickson D W, Davies P, Bevona C, Van Hoeven K H, Factor S M, Grober E, Aronson M K, Crystal H A (1994) Hippocampal sclerosis: a common pathological feature of dementia in very old (>80 years of age) humans. Acta Neuropathol 88: 212-221.
Dormann D, Capell A, Carlson A M, Shankaran S S, Rodde R, Neumann M, Kremmer E, Matsuwaki T,
Duque S, Joussemet B, Riviere C, Marais T, Dubreil L, Douar A M, Fyfe J, Moullier P, Colle M A, Barkats M (2009) Intravenous administration of self-complementary AAV9 enables transgene delivery to adult motor neurons. Mol Ther 17: 1187-1196.
Eisen A (2009) Amyotrophic lateral sclerosis: A 40-year personal perspective. J Clin Neurosci 16: 505-12.
Feiguin F, Godena V K, Romano G, D'Ambrogio A, Klima R, Baralle FE (2009) Depletion of TDP-43 affects Drosophila motoneurons terminal synapsis and locomotive behavior. FEBS Lett 583: 1586-1592.
Filimonenko, M, Stuffers, S, Raiborg, C, Yamamoto, A, Maierød, L, Fisher, E M et al. (2007). Functional multivesicular bodies are required for autophagic clearance of protein aggregates associated with neurodegenerative disease. J. Cell Biol. 179: 485-500.
Foust K D, Nurre E, Montgomery C L, Hernandez A, Chan C M, Kaspar B K (2009) Intravascular AAV9 preferentially targets neonatal neurons and adult astrocytes. Nat Biotechnol 27: 59-65.
Foust K D, Poirier A, Pacak C A, Mandel R J, Flotte T R (2008) Neonatal intraperitoneal or intravenous injections of recombinant adeno-associated virus type 8 transduce dorsal root ganglia and lower motor neurons. Human Gene Ther 19: 61-69.
Geser F, Brandmeir N J, Kwong L K, Martinez-Lage M, Elman L, McCluskey L, Xie S X, Lee V M, Trojanowski J Q (2008) Evidence of multisystem disorder in whole-brain map of pathological TDP-43 in amyotrophic lateral sclerosis. Arch Neurol 65: 636-641.
Geser F, Martinez-Lage M, Kwong L K, Lee V M, Trojanowski J Q (2009a) Amyotrophic lateral sclerosis, frontotemporal dementia and beyond: the TDP-43 diseases. J Neurol 256: 1205-1214.
Geser F, Martinez-Lage M, Robinson J, Uryu K, Neumann M, Brandmeir N J, Xie S X, Kwong L K, Elman L, McCluskey L, Clark C M, Malunda J, Miller B L, Zimmerman E A, Qian J, Van D, V, Grossman M, Lee V M, Trojanowski J Q (2009) Clinical and pathological continuum of multisystem TDP-43 proteinopathies. Arch Neurol 66: 180-189.
Geser, F, Brandmeir, N J, Kwong, L K, Martinez-Lage, M, Elman, L, McCluskey, L et al. (2008). Evidence of multisystem disorder in whole-brain map of pathological TDP-43 in amyotrophic lateral sclerosis. Arch Neurol 65: 636-641.
Gitcho M A, Baloh R H, Chakraverty S, Mayo K, Norton J B, Levitch D, Hatanpaa K J, White C L, III, Bigio E H, Caselli R, Baker M, Al Lozi M T, Morris J C, Pestronk A, Rademakers R, Goate A M, Cairns N J (2008) TDP-43 A315T mutation in familial motor neuron disease. Ann Neurol 63: 535-538.
Gitcho M A, Bigio E H, Mishra M, Johnson N, Weintraub S, Mesulam M, Rademakers R, Chakraverty S, Cruchaga C, Morris J C, Goate A M, Cairns N J (2009) TARDBP 3′-UTR variant in autopsy-confirmed frontotemporal lobar degeneration with TDP-43 proteinopathy. Acta Neuropathol, in press.
Gray S J, McCown T J, Samulski R J (2009) Global gene delivery to the central nervous system via adenoassociated virus vectors administered intravenously. Program no. 643.6.2009 Abstract Viewer/Itinerary Planner. Washington D.C.: Society for Neuroscience. (Washington, D.C.)
Hazelett D J, Thomas A, Ramaker J, Timmermans W, Morton D (2009) Loss-of-function and gain-of-function of TDP-43 results in motor deficits in Drosophilia. Program no. 633.14.2009 Abstract Viewer/Itinerary Planner. Washington D.C.: Society for Neuroscience. (Washington, D.C.)
Hutton M. (2000) Molecular genetics of chromosome 17 tauopathies. Ann NY Acad Sci 920: 62-73.
Igaz L M, Swanson E, Winton M J, Kwong L K, Trojanowski J Q, Lee V M-L (2009) Generation and characterization of transgenic mice conditionally expressing a cytoplasmic form of human TDP-43. Program no. 146.22.2009 Abstract Viewer/Itinerary Planner. Washington D.C.: Society for Neuroscience. (Washington, D.C.)
Jaworski T, Dewachter I, Lechat B, Croes S, Termont A, Demedts D, Borghgraef P, Devijver H, Filipkowski R K, Kaczmarek L, Kugler S, Van Leuven F (2009) AAV-tau mediates pyramidal neurodegeneration by cellcycle re-entry without neurofibrillary tangle formation in wild-type mice. PLoS One 4: e7280.
Johnson B S, McCaffery J M, Lindquist S, Gitler A D. (2008) A yeast TDP-43 proteinopathy model: Exploring the molecular determinants of TDP-43 aggregation and cellular toxicity. Proc Natl Acad Sci USA. 105:6439-44.
Josephs K A, Dickson D W (2007) Hippocampal sclerosis in tau-negative frontotemporal lobar degeneration. Neurobiol Aging 28: 1718-1722.
Josephs K A, Stroh A, Dugger B, Dickson D W (2009) Evaluation of subcortical pathology and clinical correlations in FTLD-U subtypes. Acta Neuropathol 118: 349-358.
Kabashi, E, Valdmanis, P N, Dion, P, Spiegelman, D, McConkey, B J, Vande Velde, C et al. (2008). TARDBP mutations in individuals with sporadic and familial amyotrophic lateral sclerosis. Nat. Genet. 40: 572-574.
Kaspar B K, Llado J, Sherkat N, Rothstein J D, Gage F H (2003) Retrograde viral delivery of IGF-1 prolongs survival in a mouse ALS model. Science 301: 839-842.
Kleim J A, Boychuk J A, Adkins D L (2007) Rat models of upper extremity impairment in stroke. ILAR J 48: 374-384.
Klein R L, Dayton R D, Diaczynsky C G, Wang D B (2009) Pronounced microgliosis and neurodegeneration in aged rats after tau gene transfer. Neurobiol Aging.
Klein R L, Dayton R D, Henderson K M, Petrucelli L (2006a) Parkin is protective for substantia nigra dopamine neurons in a tau gene transfer neurodegeneration model. Neurosci. Lett. 401:130-135.
Klein R L, Dayton R D, Leidenheimer N J, Jansen K, Golde T E, Zweig R M (2006) Efficient neuronal gene transfer with AAV8 leads to neurotoxic levels of tau or green fluorescent proteins. Mol Ther 13: 517-527.
Klein R L, Dayton R D, Lin W L, Dickson D W (2005) Tau gene transfer, but not alpha-synuclein, induces both progressive dopamine neuron degeneration and rotational behavior in the rat. Neurobiol Dis 20: 64-73.
Klein R L, Dayton R D, Tatom J B, Diaczynsky C G, Salvatore M F (2008a) Tau expression levels from various adeno-associated virus vector serotypes produce graded neurodegenerative disease states. Eur J Neurosci 27: 1615-1625.
Klein R L, Dayton R D, Tatom J B, Henderson K M, Henning P P (2008) AAV8, 9, Rh10, Rh43 vector gene transfer in the rat brain: effects of serotype, promoter and purification method. Mol Ther 16: 89-96.
Klein R L, Dayton R D, Terry T, Vascoe C, Sunderland J J, Tainter K H (2009a) PET imaging in rats to discern temporal onset differences between 6-hydroxydopamine and tau gene vector neurodegeneration models. Brain Res. 1259:113-122.
Klein R L, Hirko A C, Meyers C A, Grimes J R, Muzyczka N, Meyer E M (2000) NGF gene transfer to intrinsic basal forebrain neurons increases cholinergic cell size and protects from age-related deficits in spatial memory in middle-aged rats. Brain Res. 875: 144-151.
Klein R L, King M A, Hamby M E, Meyer E M (2002) Dopaminergic cell loss induced by human A30P alphasynuclein gene transfer to the rat substantia nigra. Human Gene Ther. 13: 605-612.
Klein R L, Lewis M H, Muzyczka N, Meyer E M (1999) Prevention of 6-hydroxydopamine-induced rotational behavior by BDNF somatic gene transfer. Brain Res. 847:314-320.
Klein R L, Lin W L, Dickson D W, Lewis J, Hutton M, Duff K, Meyer E M, King M A (2004) Rapid neurofibrillary tangle formation after localized gene transfer of mutated tau. Am. J. Pathol. 164: 347-353.
Kraemer B C, Liachko N, Guthrie C (2009) A C. elegans model of ALS and other TDP-43 proteinopathy disorders. Program no. 304.12.2009 Abstract Viewer/Itinerary Planner. Washington D.C.: Society for Neuroscience. (Washington, D.C.)
Kumar-Singh S, Van Broeckhoven C (2007) Frontotemporal lobar degeneration: current concepts in the light of recent advances. Brain Pathol 17: 104-114.
Kwong L K, Neumann M, Sampathu D M, Lee V M, Trojanowski J Q (2007) TDP-43 proteinopathy: the neuropathology underlying major forms of sporadic and familial frontotemporal lobar degeneration and motor neuron disease. Acta Neuropathol 114: 63-70.
Lagier-Tourenne C, Cleveland D W (2009) Rethinking ALS: the FUS about TDP-43. Cell 136: 1001-1004.
Laird A S, Van Damme P, Van Den Bosch L, Robberecht W L (2009) Overexpression of mutant TDP-43 RNA in zebrafish embryos leads to a motor neuron phenotype. Program no. 633.7.2009 Abstract Viewer/Itinerary Planner. Washington D.C.: Society for Neuroscience. (Washington, D.C.)
Leverenz, J B, Yu, C E, Montine, T J, Steinbart, E, Bekris, L M, Zabetian, C, Kwong L K, Lee V M, Schellenberg G D, Bird T D (2007). A novel progranulin mutation associated with variable clinical presentation and tau, TDP43 and alpha-synuclein pathology. Brain 130: 1360-1374.
Liu Y, Studzinski C, Beckett T, Guan H, Hersh M A, Murphy M P, Klein R, Hersh L B (2009) Expression of neprilysin in skeletal muscle reduces brain amyloid burden in a transgenic mouse model of Alzheimer's disease. Mol. Ther., in press.
Lowenstein P R (2009) Crossing the rubicon. Nat. Genet. 27: 42-44.
Mackenzie I R, Bigio E H, Ince P G, Geser F, Neumann M, Cairns N J, Kwong L K, Forman M S, Ravits J, Stewart H, Eisen A, McClusky L, Kretzschmar H A, Monoranu C M,
Highley J R, Kirby J, Siddique T, Shaw P J, Lee V M, Trojanowski J Q (2007) Pathological TDP-43 distinguishes sporadic amyotrophic lateral sclerosis from amyotrophic lateral sclerosis with SOD1 mutations. Ann Neurol 61: 427-434.
Manfredsson F P, Rising A C, Mandel R J (2009) AAV9: a potential blood-brain barrier buster. Mol Ther 17: 403-405.
McCarty D M, Dirosario J, Gulaid K, Muenzer J, Fu H (2009) Mannitol-facilitated CNS entry of rAAV2 vector significantly delayed the neurological disease progression in MPS IIIB mice. Gene Ther, in press.
Mochizuki, H, Hayakawa, H, Migita, M, Shibata, M, Tanaka, R, Suzuki, A et al. (2001). An AAV-derived Apaf-1 dominant negative inhibitor prevents MPTP toxicity as antiapoptotic gene therapy for Parkinson's disease. Proc Natl Acad Sci USA 98: 10918-10923.
Moisse K, Mepham J, Volkening K, Welch I, Hill T, Strong MJ (2009a) Cytosolic TDP-43 expression following axotomy is associated with caspase 3 activation in NFL(−/−) mice: Support for a role for TDP-43 in the physiological response to neuronal injury. Brain Res, in press.
Moisse K, Volkening K, Leystra-Lantz C, Welch I, Hill T, Strong MJ (2009) Divergent patterns of cytosolic TDP-43 and neuronal progranulin expression following axotomy: implications for TDP-43 in the physiological response to neuronal injury. Brain Res 1249: 202-211.
Myers A J, Pittman A M, Zhao A S, Rohrer K, Kaleem M, Marlowe L, Lees A, Leung D, McKeith I G, Perry R H, Morris C M, Trojanowski J Q, Clark C, Karlawish J, Arnold S, Forman M S, Van D, V, de Silva R, Hardy J (2007) The MAPT H1c risk haplotype is associated with increased expression of tau and especially of 4 repeat containing transcripts. Neurobiol Dis 25: 561-570.
Nandi K N, Knight D S, Beal J A (1993) Spinal neurogenesis and axon projection: a correlative study in the rat. J Comp Neurol 328: 252-262.
Neary, D, Snowden, J S and Mann, D M (2000). Classification and description of frontotemporal dementias. Ann N Y Acad Sci 920: 46-51.
Neumann M (2009) Molecular Neuropathology of TDP-43 Proteinopathies. Int J Mol Sci 10: 232-246.
Neumann M, Rademakers R, Roeber S, Baker M, Kretzschmar H A, Mackenzie I R (2009) Frontotemporal lobar degeneration with FUS pathology. Brain, in press.
Neumann M, Sampathu D M, Kwong L K, Truax A C, Micsenyi M C, Chou T T, Bruce J, Schuck T, Grossman M, Clark C M, McCluskey L F, Miller B L, Masliah E,
Mackenzie I R, Feldman H, Feiden W, Kretzschmar H A, Trojanowski J Q, Lee V M (2006) Ubiquitinated TDP-43 in frontotemporal lobar degeneration and amyotrophic lateral sclerosis. Science 314: 130-133.
Neumann, M (2008). Pathological spectrum of frontotemporal lobar degeneartion. Dement Geriatr Cogn Disord 26 S 1:1.
Nishihira Y, Tan C F, Onodera O, Toyoshima Y, Yamada M, Morita T, Nishizawa M, Kakita A, Takahashi H. (2008) Sporadic amyotrophic lateral sclerosis: two pathological patterns shown by analysis of distribution of TDP-43-immunoreactive neuronal and glial cytoplasmic inclusions. Acta Neuropathol. 116:169-82.
Nonaka T, Kametani F, Arai T, Akiyama H, Hasegawa M (2009) Truncation and pathogenic mutations facilitate the formation of intracellular aggregates of TDP-43. Hum Mol Genet, in press.
Osinde M, Clavaguera F, May-Nass R, Tolnay M, Dev KK (2008) Lentivirus Tau (P301S) expression in adult amyloid precursor protein (APP)-transgenic mice leads to tangle formation. Neuropathol Appl Neurobiol 34: 523-531.
Paquet D, Bhat R, Sydow A, Mandelkow E M, Berg S, Hellberg S, Falting J, Distel M, Koster R W, Schmid B, Haass C (2009) A zebrafish model of tauopathy allows in vivo imaging of neuronal cell death and drug evaluation. J Clin Invest 119: 1382-1395.
Passini M A, Bu J, Roskelley E M, Richards A M, Sardi S P, O'Riordan C R, Klinger K W, Shihabuddin L S, Cheng S H (2009) CNS delivery of human SMN1 by adeno-associated virus is highly efficacious in a mouse model of spinal muscular atrophy type I. Program no. 49.3.2009 Abstract Viewer/Itinerary Planner. Washington D.C.: Society for Neuroscience. (Washington, D.C.)
Paxinos G, Watson C, (1998) The rat brain in stereotaxic coordinates, 4th edn. Academic Press: San Diego, Calif.
Pirici, D, Vandenberghe, R, Rademakers, R, Dermaut, B, Cruts, M, Vennekens, K et al. (2006). Characterization of ubiquitinated intraneuronal inclusions in a novel Belgian frontotemporal lobar degeneration family. J Neuropathol Exp Neurol 65: 289-301.
Reimsnider S, Manfredsson F P, Muzyczka N, Mandel R J (2007) Time course of transgene expression after intrastriatal psuedotyped rAAV2/1, rAAV2/2, rAAV2/5, and rAAV2/8 transduction in the rat. Mol. Ther. 15: 1504-1511.
Rohn T T (2008) Caspase-cleaved TAR DNA-binding protein-43 is a major pathological finding in Alzheimer's disease. Brain Res 1228: 189-98.
Shankaran S S, Capell A, Hruscha A T, Fellerer K, Neumann M, Schmid B, Haass C (2008) Missense mutations in the progranulin gene linked to frontotemporal lobar degeneration with ubiquitin-immunoreactive inclusions reduce progranulin production and secretion. J Biol Chem 283: 1744-1753.
Singleton A B, Farrer M, Johnson J, Singleton A, Hague S, Kachergus J, Hulihan M, Peuralinna T, Dutra A, Nussbaum R, Lincoln S, Crawley A, Hanson M, Maraganore D, Adler C, Cookson M R, Muenter M, Baptista M, Miller D, Blancato J, Hardy J, Gwinn-Hardy K (2003) alpha-Synuclein locus triplication causes Parkinson's disease. Science 302: 841.
Smittkamp S E, Brown J W, Stanford J A (2008) Time-course and characterization of orolingual motor deficits in B6SJL-Tg (SOD1-G93A)1Gura mice. Neuroscience 151: 613-621.
Sreedharan J, Blair I P, Tripathi V B, Hu X, Vance C, Rogelj B, Ackerley S, Durnall J C, Williams K L, Buratti E, Baralle F, de Belleroche J, Mitchell J D, Leigh P N, Al Chalabi A, Miller C C, Nicholson G, Shaw C E (2008) TDP-43 mutations in familial and sporadic amyotrophic lateral sclerosis. Science 319: 1668-1672.
Stallings N R, Puttaparthi K, Luther C M, Elliott J L (2009) Generation and characterization of wild-type and mutant TDP-43 transgenic mice. Program no. 633.19.2009 Abstract Viewer/Itinerary Planner. Washington D.C.: Society for Neuroscience. (Washington, D.C.)
Stanford J A, Vorontsova E, Surgener S P, Gerhardt G A, Fowler S C (2003) Aged Fischer 344 rats exhibit altered orolingual motor function: relationships with nigrostriatal neurochemical measures. Neurobiol Aging 24: 259-266.
Tatom J B, Wang D B, Dayton R D, Skalli O, Hutton M L, Dickson D W, Klein R L (2009) Mimicking aspects of frontotemporal lobar degeneration and Lou Gehrig's disease in rats via TDP-43 overexpression. Mol Ther 17: 607-613.
Tomik B, Guiloff R J (2008) Dysarthria in amyotrophic lateral sclerosis: a review. Amyotroph Lateral Scler 14: 1-12.
Ubhi K, Rockenstein E, Doppler E, Mante M, Adame A, Patrick C, Trejo M, Crews L, Paulino A, Moessler H, Masliah E (2009) Neurofibrillary and neurodegenerative pathology in APP-transgenic mice injected with AAV2-mutant TAU: neuroprotective effects of Cerebrolysin. Acta Neuropathol 117: 699-712.
Uryu K, Nakashima-Yasuda H, Forman M S, Kwong L K, Clark C M, Grossman M, Miller B L, Kretzschmar H A, Lee V M, Trojanowski J Q, Neumann M (2008) Concomitant TAR-DNA-binding protein 43 pathology is present in Alzheimer disease and corticobasal degeneration but not in other tauopathies. J Neuropathol Exp Neurol 67: 555-564.
Van Damme P (2009) Zebrafish model of TDP-43 and progranulin. Third International Research Workshop on Frontotemporal Dementia in ALS, June 21-25, London, Ontario.
Wang I F, Wu L S, Chang H Y, Shen C K (2008) TDP-43, the signature protein of FTLD-U, is a neuronal activity-responsive factor. J Neurochem 105: 797-806.
Watson C, Paxinos G, Kayalioglu G (2008) The spinal cord: a Christopher and Dana Reeve text and atlas. Academic Press: Sydney.
Wegorzewska I, Bell S, Cairns N J, Miller T M, Baloh R H (2009) TDP-43 mutant transgenic mice develop features of ALS and frontotemporal lobar degeneration. Proc Natl Acad Sci USA, in press.
Winton, M J, Van Deerlin, V M, Kwong, L K, Yuan, W, Wood, E M, Yu, C E et al. (2008). A90V TDP-43 variant results in the aberrant localization of TDP-43 in vitro. FEBS Lett 582: 2252-2256.
Xu Y, Zhang Y J, Crook RJP, Yu C, Hutton M, Dickson D W, McGowan E, Lewis J, Petrucelli L (2008) Generation and characterization of human TDP-43 transgenic mice. Program no. 638.2.2008 Abstract Viewer/Itinerary Planner. Washington D.C.: Society for Neuroscience. (Washington, D.C.)
Yamanouchi K, Nishihara M, Haass C (2009) Proteolytic processing of TAR DNA binding protein-43 by caspases produces C-terminal fragments with disease defining properties independent of progranulin. J Neurochem 110: 1082-1094.
Zhang Y J, Xu Y F, Cook C, Gendron T F, Roettges P, Link C D, Lin W L, Tong J, Castanedes-Casey M, Ash P, Gass J, Rangachari V, Buratti E, Baralle F, Golde T E, Dickson D W, Petrucelli L (2009) Aberrant cleavage of TDP-43 enhances aggregation and cellular toxicity. Proc Natl Acad Sci USA 106: 7607-7612.
Zhang Y J, Xu Y F, Dickey C A, Buratti E, Baralle F, Bailey R, Pickering-Brown S, Dickson D, Petrucelli L (2007) Progranulin mediates caspase-dependent cleavage of TAR DNA binding protein-43. J Neurosci 27: 10530-10534.
Zhou H, Huang C, Xia X, (2009) Overexpression of mutated human TDP-43 gene caused progressive neurodegeneration in transgenic rats. Program no. 304.14.2009 Abstract Viewer/Itinerary Planner. Washington D.C.: Society for Neuroscience. (Washington, D.C.)

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth, and follows in the scope of the claims.

Other embodiments are within the claims.

1. An assay which models a human neurodegenerative disease in a non-human animal said assay comprises:
a) introducing an expressible gene construct comprising the TDP-43 gene into a viable non-human animal under conditions which result in expression of the genetic products of the TDP-43 gene; and
b) and measuring disease markers produced thereby which markers are associated with a neurodegenerative disease.

2. The assay according to claim 1 wherein the expressible gene construct is introduced into the viable non-human animal by a method selected from the group consisting of direct injection, intravenous injection, and infusion techniques of the construct into that portion of the brain associated with a neurodegenerative disease.

3. (canceled)

4. (canceled)

5. A method for determining the efficacy of a putative treatment regimen for a neurodegenerative disease which method comprises:
a) selecting a non-human animal or a set of non-human animals to which an expressible gene construct comprising the TDP-43 gene has been introduced under conditions which result in expression of the genetic products of the TDP-43 gene wherein, after expression, said animal or animals exhibit(s) one or more markers for a neurodegenerative disease;
b) selecting a putative treatment regimen for the neurodegenerative disease and administering said treatment to a non-human animal or set of non-human animals set forth in a) above; and
c) evaluating the change in markers relative to a non-human animal or set of non-human animals set forth in a) to which the putative treatment for the neurodegenerative disease has not been administered.

6. A construct comprising a promoter, a vector or plasmid and a TDP-43 gene wherein the construct is capable of expressing the genetic products encoded by the gene in vivo.

7. The assay of claim 1 wherein the severity of the disease is modulated by modulating the concentration of the gene construct introduced.

8. The assay according to claim 1 wherein said disease is selected from the group consisting of frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS).

9. The assay according to claim 1 wherein said gene is transferred into the rodent using an adeno-associated virus.

10. A rodent animal produced by the assay according to claim 1.

11. A method for inducing histological, functional or behavioral changes in a rodent which comprises somatic administration of wild-type of human TDP-43 directly to the brain of said rodent.

12. (canceled)

13. (canceled)

14. A method for producing a non-human animal model of a human disease which comprises transferring the human TDP-43 gene or at least one mutant form of TDP-43 together with the human SOD, tau and/or α-synuclein genes into the appropriate tissue of a living rodent under conditions which result in the expression of said wild-type or mutant TDP-43 and SOD, tau and/or α-synuclein genes.

15. (canceled)

16. The method according to claim 14 wherein said human disease is selected from the group consisting of frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS).

17. (canceled)

18. A rodent animal produced by the method of claim 14.

19. A method for inducing histological, functional or behavioral changes in a rodent model which comprises somatic administration of wild-type or mutant human TDP-43 and SOD, tau and/or a-synuclein directly to the brain of said rodent.

20. (canceled)

21. (canceled)

22. A method for producing a non-human animal model of a human disease which comprises transferring the human TDP-43 gene or at least one mutant form of TDP-43 together with one or more gene(s) that produces a gene product(s) which interacts with the wild-type or mutant TDP-43 gene into the appropriate tissue of a living rodent under conditions which result in the expression of said wild-type or mutant TDP-43 and TDP-43 interacting gene(s).

23. (canceled)

24. The method according to claim 23 wherein said human disease is selected from the group consisting of frontotemporal dementia, frontotemporal lobar degeneration with ubiquitin-positive inclusions (FTLD-U), non-Alzheimer's dementia, dementia lacking distinctive histopathology, FTLD with motor neuron disease and amyotrophic lateral sclerosis (ALS).

25. (canceled)

26. A rodent animal produced by the method of claim 22.

27. A method for inducing histological, functional or behavioral changes in a rodent model which comprises somatic administration of wild-type or mutant human TDP-43 and one or more gene(s) that produces a gene product(s) which interacts with the wild-type or mutant TDP-43 gene directly to the brain of said rodent.

28. (canceled)

29. (canceled)

30. A method of identifying gene therapies consisting of (a) transferring one or more gene(s) that produces a gene product(s) which interacts with the wild-type or mutant TDP-43 gene and either a wild-type or mutant human TDP-43 gene into a rodent; (b) determining changes in histological, functional, or behavioral activity comparing the wild-type or mutant TDP-43 transgenic animals to non-transgenic animals; and (c) identifying the TDP-43 interacting gene(s) that prevents or reduces said changes in histological, functional or behavioral activity.

31. (canceled)

32. A method of identifying gene(s) that can be used as diagnostics consisting of (a) transferring one or more gene(s) that produces a gene product(s) which interacts with the wild-type or mutant TDP-43 gene and either a wild-type or mutant human TDP-43 gene into a rodent; (b) determining changes in histological, functional, or behavioral activity comparing the wild-type or mutant TDP-43 transgenic animals to non-transgenic animals; (c) identifying the TDP-43 interacting gene(s) that enhances said changes in histological, functional or behavioral activity; and (d) demonstrating an increase in said TDP-43 interacting gene(s) in human disease.

33. A formulation comprising an aqueous buffered solution which comprises an expressible gene construct comprising the TDP-43 gene, wherein the formulation is suitable for injection, infusion and/or intravenous delivery into a non-human animal.

34. A formulation according to claim 33 which formulation is suitable for intravenous administration of a construct to the brain of an animal wherein said formulation comprises
a) a saline injection buffer,
b) a construct comprising a non-human animal or a set of non-human animals to which an expressible gene construct comprising the TDP-43 gene has been introduced under conditions which result in expression of the genetic products of the TDP-43 gene wherein, after expression, said animal or animals exhibit(s) one or more markers for a neurodegenerative disease, and
c) optionally a blood brain barrier penetrant to facilitate passage of the construct through the blood brain barrier of the animal.

35. (canceled)

36. The formulation of claim 34 wherein the blood brain barrier penetrant comprises mannitol.
